(19) United States
(12) Patent Application Publication
(10) Pub. No.: US 2023/0168198 A1
(43) Pub. Date: Jun. 1, 2023

(54) SYSTEMS AND METHODS FOR IMAGING OF REAL-TIME NUCLEIC ACID AMPLIFICATION TESTS (NAATS)

(71) Applicant: University of Washington, Seattle, WA (US)

(72) Inventors: Paul Yager, Seattle, WA (US); Kamal Girish Shah, Seattle, WA (US)

(73) Assignee: University of Washington, Seattle, WA (US)

(21) Appl. No.: 17/999,803

(22) PCT Filed: Aug. 10, 2021
(86) PCT No.: PCT/US2021/045381

§ 371 Date: Nov. 23, 2022

Related U.S. Application Data
(60) Provisional application No. 63/064,650, filed on Aug. 12, 2020.

(51) Int. Cl. G01N 21/64; C12Q 1/6844

(52) U.S. Cl. CPC G01N 21/6456; C12Q 1/6844; G01N 21/6428; G01N 2021/6419; G01N 2021/6421; G01N 2021/6441; G01N 2021/6471

(57) ABSTRACT

Systems and methods for detecting a target moiety are disclosed. A system includes a substrate holder including a porous matrix. The porous matrix includes a first detectable agent and a second detectable agent. The system includes a housing, optically coupled with the substrate holder, and shaped to optically couple with a radiation source and a radiation sensor and to optically isolate the radiation source and the radiation sensor. The system includes an excitation filter, disposed in or on the housing, configured to receive excitation electromagnetic radiation from the radiation source and to transmit a first portion of the excitation electromagnetic radiation to the porous matrix. The system also includes an emission filter, disposed in or on the housing, configured to receive emitted fluorescence electromagnetic radiation from the porous matrix and to transmit a second portion of the emitted fluorescence electromagnetic radiation, the second portion being different from the first portion.

### CROSS-REFERENCE TO RELATED APPLICATION

The present application claims the benefit of co-pending U.S. Provisional Patent Application No. 63/064,650, filed Aug. 12, 2020, the content of which is incorporated by reference in its entirety.

### STATEMENT OF GOVERNMENT LICENSE RIGHTS

This invention was made with Government support under Grant No. HR0011-11-2-0007, awarded by the Defense Advanced Research Projects Agency, and Grant No. DGE-1256082, awarded by the National Science Foundation. The Government has certain rights in the invention.

### BACKGROUND

Infectious disease diagnostic tests are increasingly intended for the point of care, including clinics or even the home. Many point-of-care assays for infectious diseases leverage the format of extant home-based assays, such as those for glucose, pregnancy, or HIV. Typically, these as-says are paper-based lateral flow tests due to their minimal cost and time to result, but these are inappropriate for many infectious diseases due to poor limits of detection. Nucleic acid amplification tests (NAATs) are a promising class of laboratory and clinical assays that have low limits of detection and short times to result by imaging amplification reactions in real time, but these often require complex user steps or bulky instruments.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Systems, devices, and methods for detecting a target moiety in a sample are described. In an aspect, the present disclosure provides a system for detecting the target moiety in a sample may include a substrate holder including a porous matrix. The porous matrix may include a first detectable agent configured to selectively couple to a first target moiety and to emit a first detectable signal upon fluorescence of the first detectable agent. The porous matrix may also include a second detectable agent configured to selectively couple to a second target moiety and to emit a second detectable signal different than the first detectable signal upon fluorescence of the second detectable agent. The system may include a housing, optically coupled with the substrate holder, and shaped to optically couple with a radiation source and a radiation sensor and to optically isolate the radiation source and the radiation sensor. The system may include an excitation filter, disposed in or on the housing, configured to receive excitation electromagnetic radiation from the radiation source and to transmit a first portion of the excitation electromagnetic radiation to the porous matrix. The system may also include an emission filter, disposed in or on the housing, configured to receive emitted fluorescence electromagnetic radiation from the porous matrix and to transmit a second portion of the emitted fluorescence electromagnetic radiation, the second portion being different from the first portion.

In some embodiments, the excitation filter includes a multiple-passband filter, and the first portion includes two non-contiguous excitation energy ranges corresponding to a first excitation wavelength range of the first detectable agent and a second excitation wavelength range of the second detectable agent, respectively. The emission filter may be or include a second multiple-passband filter, and the second portion includes two non-contiguous emitted fluorescence energy ranges corresponding to the first detectable signal and the second detectable signal, respectively. The two non-contiguous emitted fluorescence energy ranges may include a first band in a wavelength range from about 500 nm to about 550 nm and a second band in a range from about 600 nm to about 650 nm. The excitation electromagnetic radiation may be characterized by a continuous emission intensity distribution within a wavelength range from about 400 nm to about 700 nm. The emitted fluorescence electromagnetic radiation may be characterized by a biplexed intensity distribution comprising the first detectable signal and the second detectable signal.

In some embodiments, the porous matrix further includes reagents to amplify a target nucleic acid molecule and a positive control nucleic acid molecule. The first detectable agent may be or include a probe of an amplicon of the positive control nucleic acid molecule. The second detectable agent may be or include a probe of an amplicon of the target nucleic acid molecule. The porous matrix may be or include non-woven glass fiber. The first detectable agent and the second detectable agent may be colocalized on the porous matrix.

In some embodiments, the system may further include an electronic device. The electronic device may include the radiation source and the radiation sensor. The excitation filter and the emission filter may be positioned to optically couple with the radiation source and the radiation sensor, respectively. The electronic device may be a smart phone. The radiation source may be an electronic flash. The radiation sensor may be a camera.

In some embodiments, the system further includes a controller including one or more processors and a non-transitory computer readable memory storing executable instructions that, when executed by the one or more processors, cause the one or more processors to execute operations. The operations may include generating the excitation electromagnetic radiation using the radiation source, the radiation source being optically coupled with the excitation filter to transmit the first portion of the excitation electromagnetic radiation to the porous matrix. The operations may include detecting the first detectable signal and the second detectable signal using the second portion of the emitted fluorescence electromagnetic radiation received by the radiation sensor via the emission filter. The operations may also include determining a differential emission value using the first detectable signal and the second detectable signal. Determining the differential emission value may include generating a first gamma-corrected signal based on the first detectable signal and a second gamma-corrected signal based on the second detectable signal. Determining the differential emission value may also include determining a difference between the second gamma-corrected measurement and the first gamma-corrected measurement. The substrate holder may further include an electronic heating circuit thermally coupled with the porous matrix. The electronic heating circuit may be configured to heat the porous matrix. The non-transitory computer readable memory may store further instructions that, when executed by one or more processors of the system, cause the one or more processors to execute operations including heating the porous matrix to a temperature and for a period of time sufficient to amplify a target nucleic acid above a limit of detection. The limit of detection may correspond to a differential emission value greater than zero.

A method of detecting the target moiety in a sample includes generating excitation electromagnetic radiation using a radiation source, the radiation source being optically coupled with an excitation filter to transmit a first portion of the excitation electromagnetic radiation to a porous matrix, the porous matrix includes nucleic acid amplification reagents, a first detectable agent, and a second detectable agent. The method includes generating an emission signal including a first detectable signal from the first detectable agent and a second detectable signal from the second detectable agent using a second portion of emitted fluorescence electromagnetic radiation received at an optical sensor via an emission filter, the second portion being different from the first portion. The method also includes determining a differential emission value using the emission signal.

In some embodiments, the method includes heating the porous matrix to a temperature for a period of time sufficient to amplify a target nucleic acid above the limit of detection. The period of time may be in a range from about 20 minutes to about 40 minutes. The temperature may be in a range from about 300 K to about 350 K. The method may include generating a plurality of measurements of emission, including the measurement and generating a plurality of differential emission intensity values, including the differential emission intensity value, using the plurality of measurements of emission.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram illustrating an example system for detecting a target moiety in a sample, in accordance with one embodiment of the present disclosure.

FIG. 2 is a schematic diagram illustrating an example substrate holder including a porous matrix, in accordance with one embodiment of the present disclosure.

FIG. 3 is a schematic diagram illustrating an example housing to receive a radiation source and a radiation sensor, in accordance with one embodiment of the present disclosure.

FIG. 4 is a schematic diagram illustrating optical interrogation of an example bi-plexed isothermal strand displacement amplification reaction, in accordance with one embodiment of the present disclosure.

FIG. 5A is an example graph illustrating optical excitation spectra of the example system of FIG. 1, in accordance with one embodiment of the present disclosure.

FIG. 5B is an example graph illustrating detectable signals in the example system of FIG. 1, in accordance with one embodiment of the present disclosure.

FIG. 6A is an example graph illustrating exemplary normalized differential fluorescence spectra of the example system of FIG. 1, in accordance with one embodiment of the present disclosure.

FIG. 6B is an example graph illustrating exemplary normalized differential fluorescence values of the example system of FIG. 1, in accordance with one embodiment of the present disclosure.

FIG. 6C is a schematic diagram illustrating example porous matrices corresponding to a subset of the exemplary values of FIGS. 6A-6B, in accordance with one embodiment of the present disclosure.

FIG. 7 is a block flow diagram illustrating an example process for detecting a target moiety in a sample, in accordance with one embodiment of the present disclosure.

FIG. 8A is a photo of the fluorescence reader, in accordance with one embodiment of the present disclosure.

FIG. 8B is a photo of 49 spots of fluorophore mixtures of Texas Red and fluorescein on glass fiber, in accordance with one embodiment of the present disclosure.

FIG. 9A is a graph illustrating the fluorophore limits of detection of the phones at NAAT-relevant conditions, in accordance with one embodiment of the present disclosure.

FIG. 9B is a graph illustrating correlation between Texas Red concentration and fluorescence in the green color channel, in accordance with one embodiment of the present disclosure.

FIG. 9C is a graph illustrating a slight negative correlation between fluorescein concentration and fluorescence in the red color channel, in accordance with one embodiment of the present disclosure.

FIG. 10A is a graph illustrating measured refractive indices of the fibers of several glass fiber materials, in accordance with one embodiment of the present disclosure.

FIG. 10B is a schematic diagram illustrating a glass fiber pad blocked with bovine serum albumin that was wetted with index-matching p-xylene, in accordance with one embodiment of the present disclosure.

FIG. 10C is a schematic diagram illustrating glass fiber pads blocked with bovine serum albumin that were wetted with water showing evaporation and condensation after 60 minutes of heating, in accordance with one embodiment of the present disclosure.

FIG. 10D is a graph illustrating quantifying the heated pads' fluorescence intensity, in accordance with one embodiment of the present disclosure.

FIG. 11A is a schematic diagram illustrating development of fluorescence in an example negative porous matrix and an example positive porous matrix, in accordance with one embodiment of the present disclosure.

FIG. 11B is an example graph illustrating exemplary normalized differential fluorescence values as a function of time, in accordance with one embodiment of the present disclosure.

FIG. 11C is an example graph illustrating negative and positive detection from the example system of FIG. 1, in accordance with one embodiment of the present disclosure.

### DETAILED DESCRIPTION

While illustrative embodiments have been illustrated and described, it will be appreciated that various changes can be made therein without departing from the spirit and scope of the invention.

Systems, devices, and methods for detecting a target moiety in a sample are described. In some embodiments, detection of the target moiety includes point-of-care assays implemented using mobile electronic devices that have been modified to interrogate a substrate with electromagnetic radiation and to measure the emission elicited from detectable agents in proportion to the quantity of the target moiety in the substrate. As described in the forthcoming figures, the target moiety may be or include nucleic acids, proteins, amino acids, or other molecules of biological significance. In this way, detection of the target moiety may permit, for example, the point-of-care assay to screen for viral, bacterial, or fungal infection.

In some embodiments, biplexed fluorescent probes are excited together, for example, by colocalizing two detectable agents on the same substrate and interrogating the substrate with excitation radiation for both probes. In some embodiments, one of the detectable agents selectively binds to an amplification control, while the other detectable agent selectively binds to the target moiety. In this way, a differential emission signal may be generated through comparison of the emission of the two detectable agents. Advantageously, generating the differential emission signal, rather than directly measuring the emission from the detectable agent bound to the target moiety, limits the influence of substrate condition and controls for physical factors, such as microfluidic failure in sample loading or heater failure. As such, a differential emission value may be defined, such that a positive value indicates the presence of the target moiety, a negative value indicates the absence of the target moiety, and a near-zero value indicates a test failure.

In an illustrative example, a system for the detection of the target moiety includes a glass fiber porous matrix, loaded with dried and lyophilized reagents for a nucleic acid amplification test (NAAT). The NAAT reagents include two detectable agents and amplification enzymes (e.g., polymerase). One of the detectable agents includes a green fluorescent (e.g., fluorescein) probe prepared to selectively bind to DNA used as an amplification control. The other detectable agent includes a red fluorescent (e.g., Texas Red) probe prepared to selectively bind to methicillin-resistant S. aureus (MRSA) genomic DNA. A sample collected from a test subject, such as a cheek or nasal swab, is processed to prepare a liquid solution that is deposited onto one end of the glass fiber porous matrix. The sample is drawn into the porous matrix and heated for a period of time to amplify the DNA in the sample.

During the period of time, excitation radiation from a light source is used to interrogate the detectable agents, stimulating fluorescence of two detectable signals with an intensity proportional to the quantities of control DNA and MRSA DNA. The excitation radiation is generated by a continuous “white light” source (e.g., a halogen lamp) and filtered by an excitation filter that includes two passbands corresponding to the absorbance bands of the first and second detectable agents, respectively. Similarly, fluorescence emission from the porous matrix is filtered by an emission filter that also includes two passbands, corresponding to the emission spectra of the first and second detectable agents, respectively. In this way, emission from the detectable agents is collected by a radiation sensor and used to determine the differential emission signal. If, after the period of time (e.g., 45 minutes) the differential emission value is positive, the result of the test is positive.

While description of embodiments focuses on visible light NAAT tests, alternative configurations are contemplated within the scope of the present disclosure. For example, other target moieties and electromagnetic spectra are considered where differential emission spectroscopy would provide a positive control to limit the influence of substrate conditions on detection, and would further permit for a quality check of the functionality of the test. Furthermore, more than two detectable signals are contemplated, whereby a single test could include detectable agents that selectively bind to an amplification control as well as two or more target moieties. In another example, embodiments are contemplated that employ fewer than two detectable agents. For example, an amplification test may employ a single detectable agent to detect a target nucleic acid sequence without a positive control, as is done in loop-mediated isothermal amplification (LAMP) assays. In some embodiments, direct measurement of the single detectable agent by a narrow-band radiation source may be done in place of differential emission spectroscopy with a broad-band radiation source, and a single-bandpass filter may be used in place of a dual-passband filter configuration. In this way, various radiation sources, radiation sensors, filter configurations, and/or detectable agents are contemplated as part of the techniques for detecting one or more target moieties in a point-of-care system.

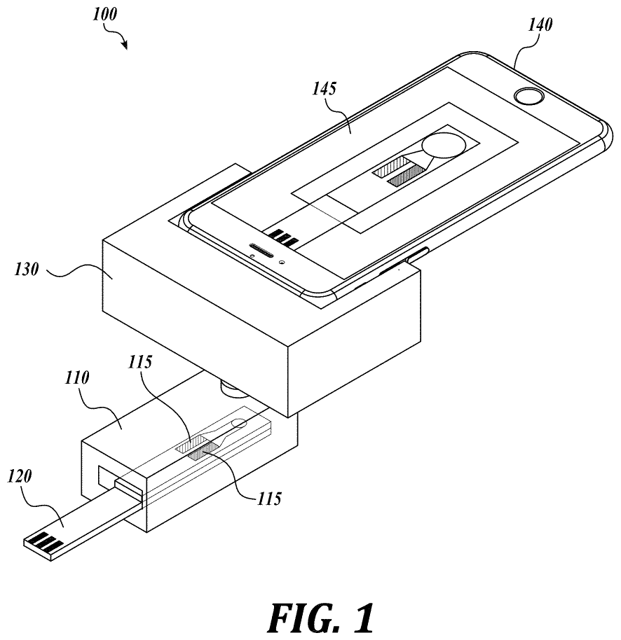

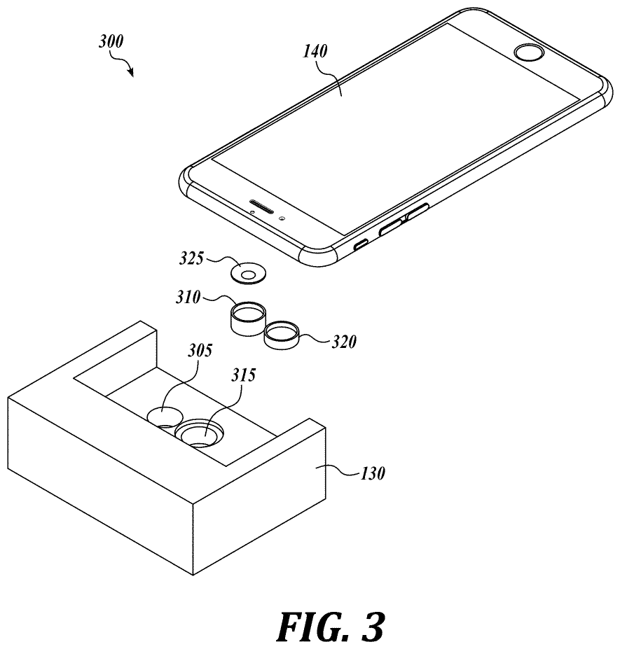

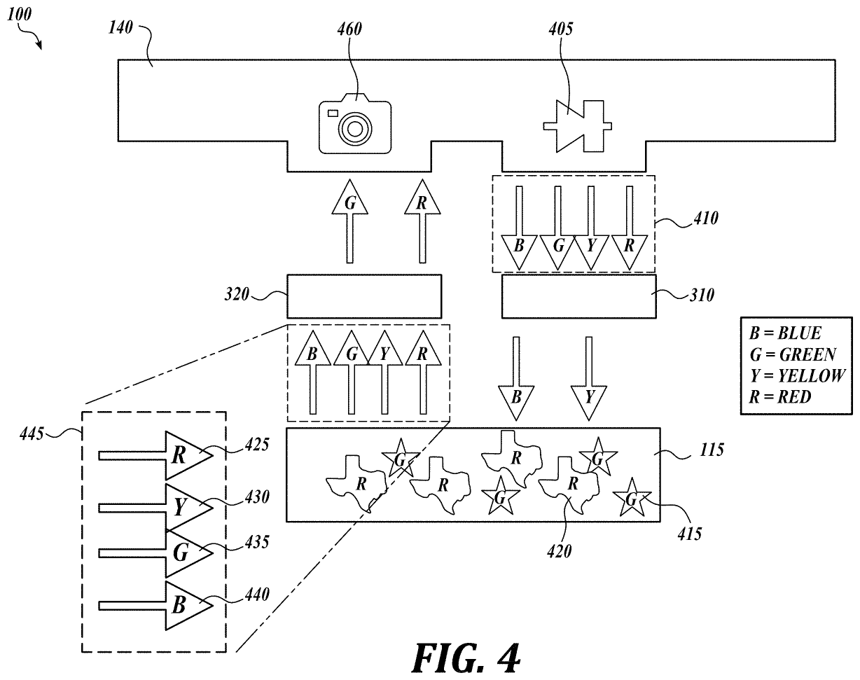

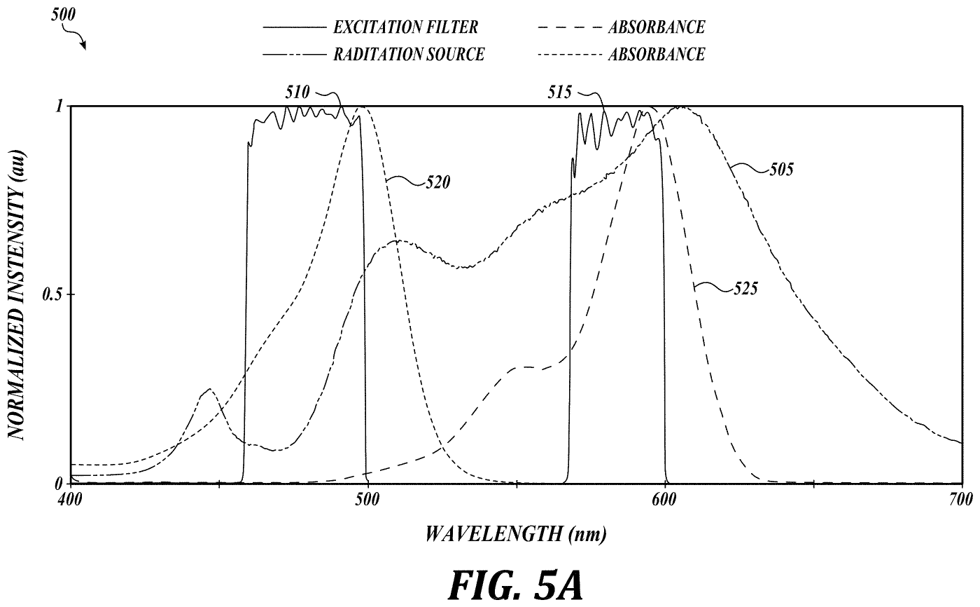

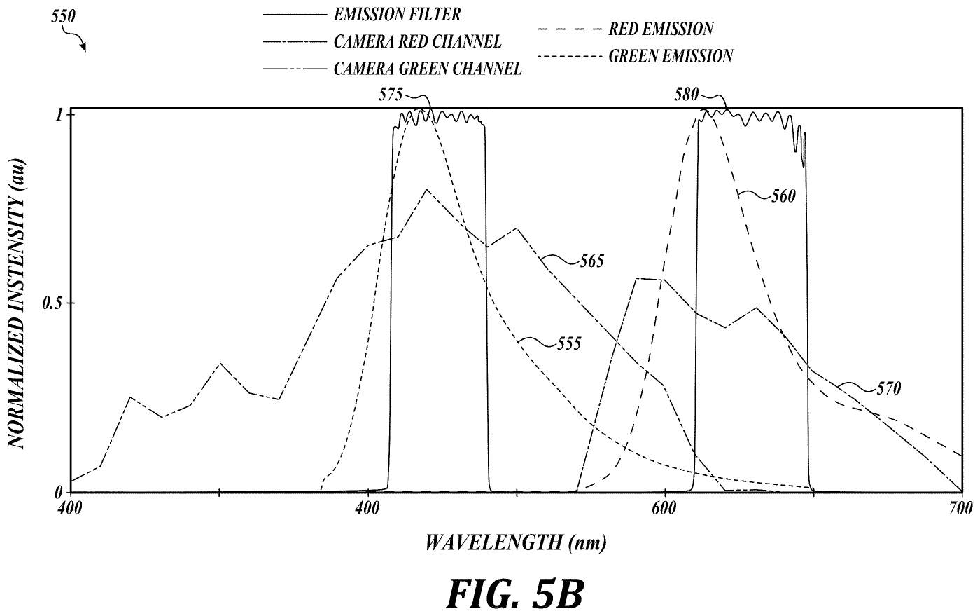

FIG. 1 is a schematic diagram illustrating an example system 100 for detecting a target moiety in a sample, in accordance with one embodiment of the present disclosure. The example system 100 includes a substrate holder 110, a housing 130, and an electronic device 140. The substrate holder 110 includes one or more porous matrix 115 pads and control and/or heating components 120. The housing 130 includes excitation and emission filters, as described in more detail in reference to FIG. 3. The electronic device 140, illustrated as a smart phone, includes a radiation source and a radiation sensor, as described in more detail in reference to FIGS. 4-5B, and a display 145.

The porous matrix 115 may be or include a scattering material including, but not limited to, a nonwoven glass fiber fabric or felt. As described in more detail in reference to Example 1, below, the scattering material may exhibit condition-dependent interaction with incident electromagnetic radiation, such that liquid saturation, solvent chemistry, and other factors may influence the signals received during an assay. To that end, the porous matrix 115 may be or include biologically compatible materials to serve as a scaffold or reservoir of amplification reagents and/or fluorescent probes.

In some embodiments, the porous matrix 115 includes a first detectable agent, as described in more detail in reference to FIG. 4. The first detectable agent is configured to selectively couple and/or bind to a first target moiety and to emit a first detectable signal upon fluorescence of the first detectable agent. In the context of a nucleic acid amplification test, the first target moiety may be a nucleic acid sequence that is used as a positive control for detecting whether the system is operating correctly. In this context, the first detectable agent may be or include a fluorescent probe (e.g., fluorescein) that has been conjugated with a binding sequence paired to the first target moiety. It is understood, however, that amplification may also be used to detect molecules of biological significance other than nucleic acids, such as proteins.

The porous matrix 115 also includes a second detectable agent, as described in more detail in reference to FIG. 4. The second detectable agent is configured to selectively couple to a second target moiety and to emit a second detectable signal different than the first detectable signal upon fluorescence of the second detectable agent. As with the first target moiety, the second target moiety may include DNA from a pathogenic organism (e.g., a virus, bacterium, or fungus) that may be detected in traces in and/or on a biological subject, such as a human. In an illustrative example, the second target moiety may be or include a methicillin-resistant S. aureus (MRSA) DNA sequence. In this context, the second detectable agent may be or include a fluorescent probe (e.g., Texas Red) that has been conjugated with a binding sequence paired to the second target moiety. In this way, the first detectable agent may be a probe of an amplicon of a positive control nucleic acid molecule. Similarly, the second detectable agent is a probe of an amplicon of a target nucleic acid molecule.

The housing 130 may optically couple with the substrate holder 110, and be shaped to optically couple with the electronic device 140 in a position corresponding to the radiation source and the radiation sensor and to optically isolate the radiation source and the radiation sensor, as described in more detail in reference to FIG. 3. Through this optical communication, excitation radiation is transmitted from the radiation source to the porous matrix 115, and emission is received from the porous matrix 115 by the radiation sensor.

In the context of example system 100, the electronic device 140 is a smart phone, the radiation source is an electronic flash, and the radiation sensor is a camera. The electronic flash and the camera are integrated into the smart phone, for example, as used to take pictures and video using native software stored on the phone. It is understood, however, that the radiation sensor and radiation source may be physically separate devices, rather than being integrated into a single electronic device 140.

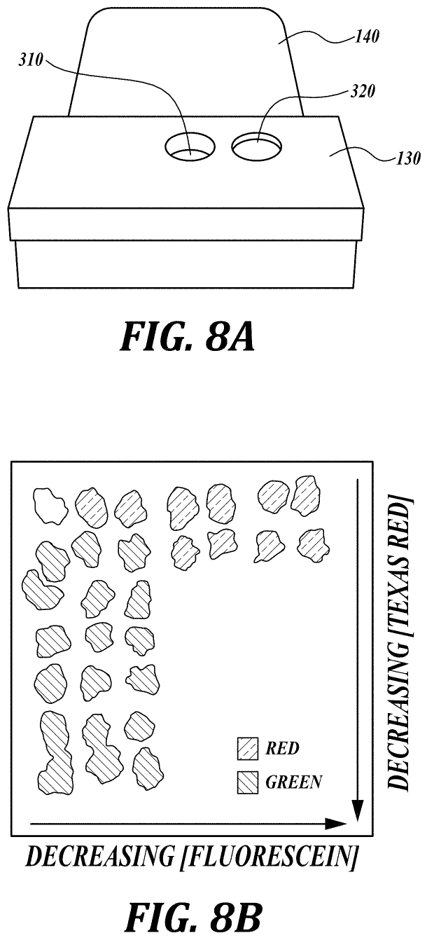

In the example system 100, the electronic device 140 includes a controller including one or more processors and a non-transitory computer readable memory. In the context of a smart phone or other computing device, the processors and non-transitory memory may be or include general purpose electronic circuitry, such as one or more CPUs and SSD “flash” memory components, storing executable instructions that, when executed by the one or more processors, cause the one or more processors to execute one or more algorithms in software. The algorithms may include operations for detecting the first and second detectable agents, for example, by generating excitation electromagnetic radiation using the radiation source, by detecting the first detectable signal and the second detectable signal using the radiation sensor, and by determining a differential emission value using the first detectable signal and the second detectable signal, as described in more detail in reference to FIG. 8.

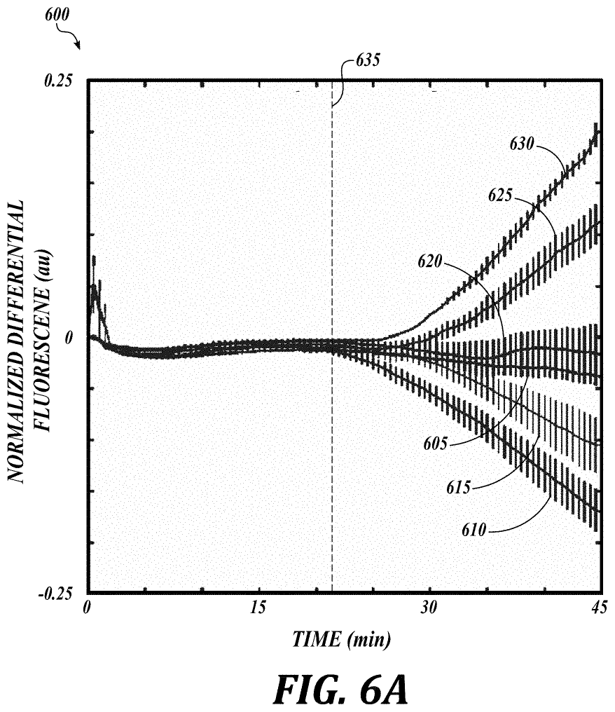

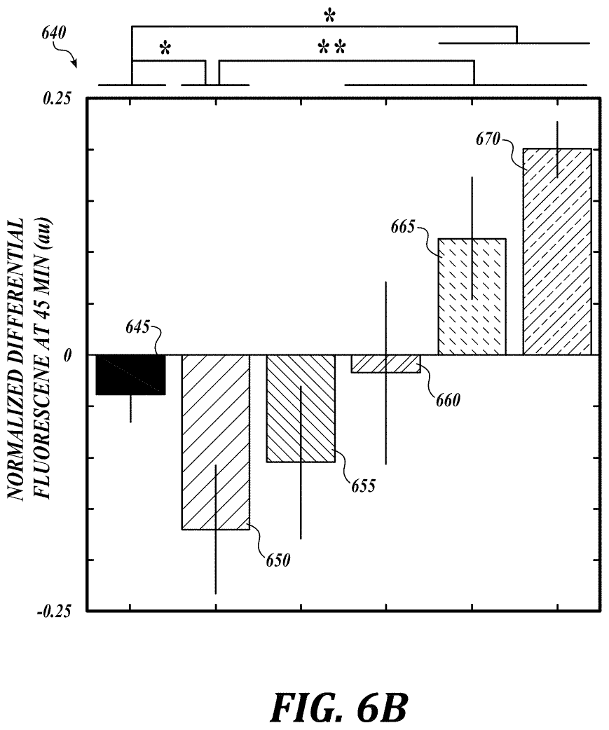

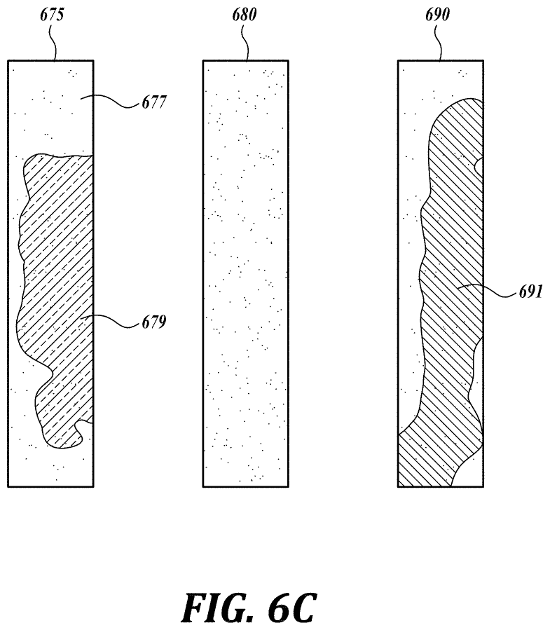

In some embodiments, the electronic device 140 includes a display 145. The algorithms in software may also include operations for generating and/or presenting the differential emission value and/or the detectable signals in various visual formats, such as bar graphs, textual messages, line graphs, scatter plots, color images, etc. For example, FIG. 1 illustrates an image being presented on the display 145. In some embodiments the electronic device 140 generates a notification via the display 145 to a user of the electronic device 140. For example, where the differential emission value indicates the presence of the second target moiety above a detection threshold, the notification may include the estimated concentration of the target moiety as well as color and/or text to indicate a positive result. The determination of the differential emission value, the detection of the target moiety, and the estimation of the concentration of the target moiety are described in more detail in reference to FIGS. 6A-8.

The software may be or include an application executed in the operating system environment of the electronic device 140, and may interface with application layers and/or hardware layers of the electronic device 140 as part of accessing/controlling the radiation source and the radiation sensor. In some embodiments, control of the radiation source is implemented by interfacing with camera software on an application layer of the electronic device 140. In some embodiments, the application may interface directly with the hardware layer of the electronic device 140, for example, where electronic device 140 is a purpose-built device for point-of-care detection testing.

Similarly, software implementing the operations described above may be implemented at least in part over a network connection in communication with one or more remote computer systems. In this context, the term “remote” describes computer systems that are physically separate from the electronic device 140. For example, remote systems may be or include computer systems (e.g., a personal computer, laptop, or tablet) electronically communicating with the electronic device 140 over a network (e.g., WIFI, local area network, private network, or the internet) or by direct pairing over nearfield communication (e.g., Bluetooth). In some embodiments, some operations, such as heating the porous matrix 115, may be executed by a computing device different from the electronic device that is in electronic communication with the substrate holder 110 (e.g., via control/heating components 120). In some embodiments, outputting operations include transmitting test results and or raw data to a remote data store, such as a distributed storage system (e.g., cloud storage) or a networked server, such that the data may be delivered to additional electronic devices.

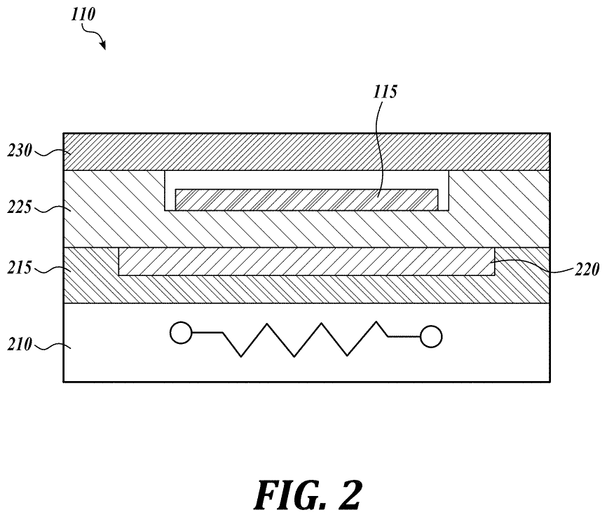

The example system 100 includes multiple porous matrix 115 pads included in the substrate holder 110. Including multiple porous matrix 115 pads permits multiple target moieties to be assayed from a single sample in parallel. Including different combinations of detectable agents in each porous matrix 115, different target moieties may be assayed. In an illustrative example, two different fluorescent probes designed to selectively bind to two different bacterial DNA sequences may be individually loaded into one of the porous matrix 115 pads, such that a sample may be assayed for the presence of both bacterial DNA sequences concurrently. In the example system 100, where a smartphone camera detects and generates the differential emission signals, the region of the image that describes the relevant porous matrix 115 may be cropped to isolate radiation emitted by the relevant detectable agent. Alternatively, each porous matrix 115 may include the same pair of detectable agents, serving to provide replicated tests for added robustness against mechanical or chemical defects in the substrate holder 110. For example, replicated testing may reduce the frequency of failure in microfluidic systems preventing a test result. As a period of time as long as 15-20 minutes may pass before a failure may be discerned, referred to as the “take-off” point, replication may significantly reduce the latency introduced by failed tests. These and other operational features of the substrate holder 110 are described in more detail in reference to FIG. 2.

FIG. 2 is a schematic diagram illustrating an example substrate holder 110 including a porous matrix 115, in accordance with one embodiment of the present disclosure. The substrate holder 110 includes an electronic heating circuit 210, a thermal coupling layer 215, an optical isolation layer 220, a retention layer 225, and a transmission window 230. The substrate holder 110 is configured to optically couple with components of the housing 130.

An example of the substrate holder 110 is described in more detail in U.S. Pat. No. 10,935,149B2, entitled, “temperature-actuated valve, fluidic device, and related methods of use.” The components of substrate holder 110 are arranged and/or configured such that the porous matrix 115 may be loaded with nucleic acid amplification reagents and detectable agents. The porous matrix 115 may be retained in position in the retention layer 225, while being interrogated by excitation radiation via the transmission window 230 during a period of time when the porous matrix 115 is being heated by the electronic heating circuit 210 via the thermal coupling layer 215.

The heating circuit 210 may be or include miniaturized heating elements, such as resistive heating circuits, localized to the lateral position of the porous matrix 115 in the substrate holder 110. The heating circuit 210 may be or include power connections such that the substrate holder 110 and the heating circuit 210 is powered by universal serial bus (e.g., 5 VDC) power, or by other readily available power sources, including but not limited to 12 VDC, 9 VDC, 3.6 VDC, 1.5 VDC, or the like. In this way, substrate holder 110 may be configured to electrically couple with a power adapter to convert line power (e.g., 120 VAC, 220 VAC) to the correct input voltage.

The heating circuit 210 may also include temperature sensors, such as microelectronic temperature sensors, to control the flow of heat into the porous matrix 115 and to maintain the temperature of the porous matrix 115 at or near an amplification setpoint. As the amplification operates by action of one or more enzymes (e.g., polymerase), the setpoint temperature may be determined as a balance between reaction rate and thermal deactivation. For example, enzymatic reaction rates may increase with temperature up to a point where the enzymes begin to denature. In this way, a temperature significantly above or below the set point may impair system performance. In some embodiments, the temperature set point is about 50° C., but may also be about 10° C. (283 K), about 15° C. (288 K), about 20° C. (293 K), about 25° C. (298 K), about 30° C. (303 K), about 35° C. (308 K), about 40° C. (413 K), about 45° C. (418 K), about 50° C. (423 K), about 55° C. (428 K), about 60° C. (433 K), about 65° C. (438 K), about 70° C. (443 K), about 75° C. (438 K), about 80° C. (443 K), or fractions or interpolations thereof. In this context, the term “about” is used to indicate a value 3-5° C. (3-5 K) on either side of the stated value.

The thermal coupling layer 215 may be or include adhesive materials exhibiting relatively high thermal conductivity. Examples of adhesive materials include, but are not limited to, double-sided thermal interface tape formed from a thermally conductive polymer or metal ribbon coated in acrylic adhesive (e.g., acrylic pressure-sensitive adhesives).

The optical isolation layer 220 may be or include a broad reflector or a broad absorber, such the thermal circuit 210 or the thermal coupling layer 215 is not detected by the radiation sensor of the electronic device 140. The optical isolation layer 220 may include a metal ribbon or other mirror surface reflective across the energy spectrum of the excitation radiation generated by the radiation source of the electronic device 140. Fluorescence in liquid solutions has a tendency to be isotropic. In this way, a reflective optical isolation layer 220 may improve diagnostic sensitivity of example system 100, for example, by redirecting signal from porous matrix 115 back to the radiation sensor.

Additionally or alternatively, the optical isolation layer 220 may include absorber material over at least a portion of the surface of the thermal coupling layer 215. Absorber material may be or include opaque polymers (e.g., low-density polyethylene), carbon tape, graphite paint, or other broad absorber across the energy spectrum of the excitation radiation generated by the radiation source of the electronic device 140. Advantageously, using an absorber can improve the quality and accuracy of the assay at least in part by reducing the portion of radiation received at the radiation sensor originating from sources other than fluorescence of the detectable agents.

The retention layer 225 may be or include chemically inert or unreactive materials or materials that are compatible with biological molecules including, but not limited to, enzymes, nucleic acids, amino acids, or proteins. In some embodiments, the retention layer 225 includes poly methyl methacrylate (PMMA), polytetrafluoroethylene (PTFE), acrylic, polycarbonate, and/or polypropylene materials. In some embodiments, the retention layer 225 is opaque across the energy spectrum of radiation or emission energies, while the transmission window 230 is transparent. The transmission window 230 may be formed from similar biologically inert materials to those of the retention layer 225. In some embodiments, the retention layer 225 and the transmission window 230 are transparent to radiation and emission energies. In this way, the retention layer 225 and the transmission window 230 together define a chamber internal to the substrate holder 110 where the porous matrix 115 may be exposed to a test sample and accurately and precisely interrogated by the electronic device 140 through the holder 130.

FIG. 3 is a schematic diagram illustrating an example housing 130 to receive a radiation source and a radiation sensor, in accordance with one embodiment of the present disclosure. The example housing 130 is an example of the housing 130 described in reference to FIG. 1, being shaped to removeably couple with the electronic device 140 described in reference to FIG. 1. The example housing 130 includes a first aperture 305 shaped to receive an excitation filter 310, a second aperture 315 shaped to receive an emission filter 320, and optics 325.

The excitation filter 310 may be disposed in or on the housing, for example, being set into the first aperture 305. In some embodiments, the first aperture 310 includes a counterbored hole formed in the housing (e.g., by molding or by machining) including a counterbore of width and depth to accept the excitation filter 310. For example, the counterbore may be shallower than the height of the excitation filter 310 or may be deeper than the height of the excitation filter 310. In some embodiments, the first aperture 305 is a through-hole passing straight through the housing 130 from the counterbore through to the opposing face of the housing 130. In some embodiments, the first aperture 305 is formed as a waveguide, reflective conduit, or refractive conduit internal to the housing, such that the outlet of the first aperture 305 is repositioned relative to the location of excitation filter 310. In this way, the excitation filter 310 may be set into the first aperture and configured to receive excitation electromagnetic radiation from the radiation source of the electronic device 140. As described in more detail in reference to FIG. 4 and FIG. 5A, excitation electromagnetic radiation includes radiation (e.g., photons) having energy meeting or exceeding the excitation energy of the detectable agents. For example, the excitation electromagnetic radiation may be generated by a continuous-spectrum source, such as a tungsten-halogen lamp, that emits at wavelengths at or above the energy threshold to excite electrons of the detectable agents into an excited state. In this way, the housing 130 may be configured to conduct the excitation electromagnetic radiation from the radiation source to the porous matrix 310.

In some embodiments, the excitation filter 310 is configured to transmit a portion of the excitation electromagnetic radiation to the porous matrix 115. For example, the excitation filter 310 may be a multiple-passband filter. In this context, a multiple-passband filter describes a filter, such as an optical bandpass filter, which selectively absorbs or blocks incident electromagnetic radiation outside two or more passbands, as described in more detail in reference to FIGS. 5A-5B. In some embodiments, the excitation filter 310 transmits two non-contiguous excitation energy ranges corresponding to a first excitation wavelength range of the first detectable agent and a second excitation wavelength range of the second detectable agent, respectively.

In some embodiments, the emission filter 320 is similarly disposed in or on the housing. As with the first aperture 305, the second aperture 315 may be or include a counterbore to receive the emission filter 320 at least partially below the surface of the housing 130, such that the emission filter 320 may be configured to receive emitted fluorescence electromagnetic radiation from the porous matrix 115 and to transmit a portion of the emitted fluorescence electromagnetic radiation to the radiation sensor. In contrast to the excitation electromagnetic radiation, the emitted fluorescence electromagnetic radiation corresponds to emission from the detectable agents, and, as such, is lower in energy than the excitation wavelength ranges used to excite the detectable agents. In this way, the emission filter 320 may be a second multiple-passband filter, filtering electromagnetic radiation received from the substrate holder 110 and/or the ambient environment by blocking incident radiation outside two non-contiguous emitted fluorescence energy ranges corresponding to the first detectable signal and the second detectable signal, respectively. In some embodiments, the corresponding passbands of the excitation filter 310 and the emission filter 320 (e.g., first detectable signal excitation passband and emission passband) are non-overlapping in energy and may be contiguous, for example, where the emission spectrum for a given detectable signal is at least partially overlapping with the absorbance spectrum of the probe, such as a fluorophore. Example spectroscopic and other optical characteristics of the emission filter, detectable signals, and the radiation sensor are described in more detail in reference to FIG. 5B.

In some embodiments, the housing 130 incorporates or includes one or more optics 325 to shape, steer, filter, or block the incident excitation electromagnetic radiation and/or the emitted fluorescence electromagnetic radiation. For example, the housing 130 illustrated in FIG. 3 includes a plano-convex lens positioned in the path of emission to at least partially collimate radiation into the radiation source of the electronic device 140. In some embodiments, the optics 325 may include multiple components to carry, reshape, and/or divert incident or emitted radiation to or from the radiation source and/or the radiation sensor. In an illustrative example, the optics may include a collecting surface overlying at least a portion of the housing 130 such that the radiation source and the excitation filter 305 may be optically coupled without direct optical alignment. Similarly, the optical collecting surface may increase the flux incident on the surface of the porous matrix 115, serving to improve the signal strength received via the emission filter 320. Similarly, the emission filter 315 may be coupled with one or more optics 325 to facilitate optically coupling the emission filter 320 with the radiation sensor (e.g., camera of electronic device 140). In some embodiments, emission-side optics 325 may be or include one or more lenses or lens assemblies to conduct emitted fluorescence electromagnetic radiation from the emission filter 320 to the expected position of the radiation sensor when the housing 130 is coupled with the electronic device 140.

In some embodiments, the optics 325 may be included as physically interchangeable assemblies, specifically configured to optically couple the respective filters to the expected position of the radiation source or radiation sensor, based on the model or design of the electronic device 140. For example, where the electronic device 140 is a smart phone, the radiation source is a flash lamp, and the radiation sensor is a camera, different models of phone typically have the camera and the flashlamp in different and widely varying positions. In this way, the filters and/or the optics 325 may be incorporated into an insert to be reversibly coupled with the housing 130 such that the housing 130, when coupled with the electronic device 140, optically couples the camera and the flash lamp of the smart phone with the porous matrix 115 of the substrate holder 110, via the housing 130.

FIG. 4 is a schematic diagram of illustrating optical interrogation of an example biplexed isothermal strand displacement amplification reaction, in accordance with one embodiment of the present disclosure. The example amplification reaction is illustrated in the context of example system 100 of FIG. 1, but it is understood that some components of example system 100 have been omitted (e.g., the substrate holder 110, the housing 130, etc.) for simplicity and to focus on optical communication between the porous matrix 115 the electronic device 140, the excitation filter 310, and the emission filter 320. As part of the optical interrogation of the example amplification reaction, a radiation source 405 of the electronic device generates excitation electromagnetic radiation 410. Disposed on the porous matrix 115 are a first detectable agent 415 and a second detectable agent 420, shown in FIG. 4 as fluorescein (“G”) and Texas Red (“R”) respectively. In the context of FIG. 4, optical interrogation includes generation and detection of four distinct wavelength ranges of visible photons. The wavelength ranges include reddish wavelengths 425, yellowish wavelengths 430, greenish wavelengths 435, and bluish wavelengths 440. Stimulation of the detectable agents 415 and 420 result in emission of a first detectable signal 450 and a second detectable signal 455 as emitted fluorescence electromagnetic radiation 445 that combines with the incident wavelengths 430 and 440 to be received by a radiation sensor 460 via the emission filter 320.

The excitation electromagnetic radiation 410 is illustrated in FIG. 4, as visible-wavelength photons covering a spectrum including wavelengths 425-440 in the bluish range, the greenish range, the yellowish range, and the reddish range. In this context, “bluish” describes a wavelength range from approximately 380 nm to 500 nm, “greenish” describes a wavelength range from approximately 490 nm to 575 nm, “yellowish” describes a wavelength range from approximately 565 nm to 625 nm, and “reddish” describes a wavelength range from approximately 615 nm to 755 nm.

In some embodiments, the radiation source 405 is a continuous light source, such as a halogen light (e.g., a Xenon flash) or a broad-spectrum light emitting diode (e.g., a white-light dual-LED). As such, the excitation radiation 410 may include radiation in an energy range that is sufficient to excite the detectable agents 415 and 420 in the porous matrix 115. As described in more detail in reference to FIG. 3, the excitation filter 310 includes multiple passbands that selectively block, absorb, or reject photons having energies outside ranges corresponding to absorbance spectra of the detectable agents, as described in more detail in reference to FIG. 5A. As illustrated, the emission filter 310 absorbs reddish wavelengths 425 and greenish wavelengths 435 and allows yellowish wavelengths 430 and bluish wavelengths 440 to pass on to the porous matrix 115. In some embodiments, the passbands of the emission filter 310 selectively transmit different combinations of the wavelengths 425-440, corresponding to the absorbance characteristics of the detectable agents being employed.

While the radiation source 405 and the radiation sensor 460 are illustrated as being incorporated into a single electronic device 140, in some cases the radiation source 405 and the radiation sensor 460 may be separate components of an optical system. In an illustrative example, the radiation source 405 and the radiation sensor may be separately integrated into the housing 130, optically coupled with the excitation filter 310 or the emission filter 320 accordingly, and controlled through a connection with an external computing device, not shown.

Advantageously, selective filtering of the excitation electromagnetic radiation 410 and the emitted fluorescence electromagnetic radiation 445 improves the accuracy and precision of the optical interrogation and reduces noise introduced into the signal detected at the radiation sensor 450. For example, the first detectable agent 415 emits greenish wavelengths 435 and the second detectable agent 430 emits reddish wavelengths 440. The characteristic emission spectra of fluorescein and Texas red are described in detail in FIG. 5B. Filtering of corresponding wavelengths from incident excitation electromagnetic radiation 410 serves to isolate the detectable signals 450 and 455 from the detectable agents 415 and 420 and boost the signal to noise ratio by reducing background noise. Similarly, selective filtering of bluish wavelengths 440 and yellowish wavelengths 430 serves to improve the accuracy of differential emission measurements where the detectable signals 450 and 455 at least partially overlap with bluish wavelengths 440 and/or yellowish wavelengths 430. In an illustrative example, the emitted fluorescence electromagnetic radiation 445 is characterized by a biplexed intensity distribution including the first detectable signal 450 and the second detectable signal 455. Biplexing describes where the detectable signals 450 and 455 at least partially overlap in wavelength and combine to form a continuous radiation energy distribution across the respective energy ranges. Biplexing may occur when the detectable agents 415 and 420 are colocalized on the porous matrix 115 and are excited by the excitation electromagnetic radiation 410. Filtration by the emission filter 320 isolates each detectable signal, by positioning each respective passband near the peak emission intensity for each respective detectable signal, as illustrated in FIG. 5B.

FIG. 5A and FIG. 5B describe example spectra corresponding to excitation electromagnetic radiation, excitation filtering, and absorbance properties of two detectable agents. While embodiments in FIG. 5A and FIG. 5B focus on optical spectra in the visible spectrum, in some embodiments at least some of the described elements are in alternative energy domains. For example, the excitation filter may transmit within a passband in the ultraviolet or infrared energy ranges. In general, an arrangement is contemplated whereby the emission filter selectively transmits excitation electromagnetic radiation in two or more noncontiguous energy ranges corresponding to absorbance spectra of the detectable agents employed to detect a target moiety and/or to serve as an amplification control. Similarly, the excitation filter selectively transmits at least a portion of emitted fluorescence electromagnetic radiation generated by the target moiety and/or the amplification control in two or more noncontiguous energy ranges.

FIG. 5A is an example graph 500 illustrating optical excitation spectra of the example system 100 of FIG. 1, in accordance with one embodiment of the present disclosure. The example graph 500 includes exemplary spectra, plotted on axes for wavelength on the abscissa and normalized intensity on the ordinate. The example graph 100 includes an emission spectrum 505 for the radiation source 405, a first passband 510 of the excitation filter 315, a second passband 515 of the excitation filter 315, a first absorbance spectrum 520 corresponding to the first detectable agent 415, a second absorbance spectrum 525 corresponding to the second detectable agent 420.

The emission spectrum 505 for the radiation source 405 represents an example intensity distribution spectrum for the excitation electromagnetic radiation 410. As illustrated, the emission spectrum 505 is characterized by a continuous emission intensity distribution within a wavelength range from about 400 nm to about 700 nm. In some embodiments, the emission spectrum 505 is characterized by a continuous emission intensity distribution across a wavelength range including energies sufficient to excite the detectable agents 415 and 420 of FIG. 4. For example, the wavelength range of the emission spectrum may include wavelengths from about 300 nm to about 700 nm, from about 300 nm to about 800 nm, from about 400 nm to about 700 nm, from about 400 nm to about 800 nm, from about 500 nm to about 700 nm, from about 500 nm to about 800 nm, from about 500 nm to about 600 nm, or interpolations thereof. While the intensity, normalized to a peak value of one and a base value near zero measured in arbitrary units (AU), is nonuniform across the wavelength range, the emission spectrum 505 includes a nonzero value of intensity across the non-contiguous energy ranges corresponding to the first passband 510 and the second passband 515.

The first passband 510, corresponding to an energy range across which the emission filter 315 is substantially transparent and at least partially overlapping the first absorbance spectrum 520, extends from about 450 nm to about 500 nm. In this context, the term “substantially” is used to indicate a value within 15% of the stated value. The excitation filter 315 attenuates incident radiation outside the first passband 510 almost completely. In some embodiments, transmission varies within the first passband 510 across a range of approximately 15% of the nominal optical density.

As with the first passband 510, the second passband 515 at least partially overlaps the second absorbance spectrum 525. In FIG. 5A, the second passband extends from about 550 nm to about 600 nm. Advantageously, the correspondence of the first passband 510 with the first absorbance spectrum 520 and the second passband 515 with the second absorbance spectrum 525 permits the detectable agents 415 and 420 to be selectively interrogated during operation of the example system 100.

FIG. 5B is an example graph 550 illustrating detectable signals in the example system 100 of FIG. 1, in accordance with one embodiment of the present disclosure. The example graph 550 includes spectra describing a first detectable signal 555, a second detectable signal 560, a camera green channel 565, a camera red channel 570, a first passband 575, and a second passband 580. As in FIG. 5A, the example graph 550 includes exemplary spectra, plotted on axes for wavelength on the abscissa and normalized intensity on the ordinate. While the description of FIG. 5B focuses on detecting the first detectable signal 555 and the second detectable signal 560 by a camera, it is understood that this is an exemplary embodiment, and that the camera described by the camera green channel 565 and the camera red channel 570 is an example of the radiation sensor 460 of FIG. 4.

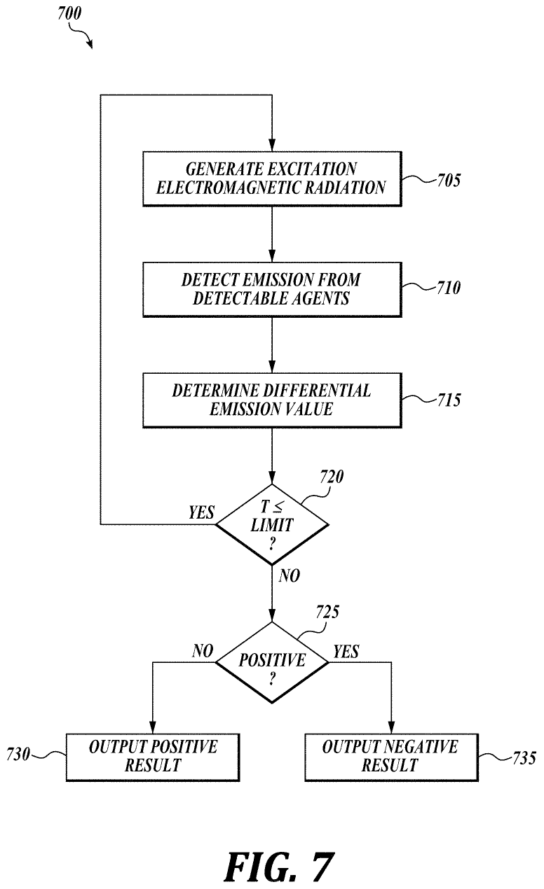

The first detectable signal 555 and the second detectable signal 560 are generated by first detectable agent 415 and second detectable agent 420, respectively. In the example system 100, the detectable signals 555 and 560 are generated by fluorescence of probes of amplicons of positive control nucleic acid molecules and amplicons of target nucleic acid molecules, respectively. As described in more detail in reference to FIGS. 6A-7C, the intensity of each detectable signal is proportional to the number of amplicons present as a function of time. The example graph 550 is normalized to the maximum value of each respective spectrum, such that proportionality information is not preserved to simplify the explanation of optical properties of the example system 100.

The example graph 550 illustrates that the first passband 575 overlaps at least a portion of the first detectable signal 555 and the camera green channel 565. Similarly, the second passband 580 overlaps at least a portion of the second detectable signal 560 and the camera red channel 570. In this way, the emission filter 320 is configured to selectively transmit the detectable signals 555 and 565 to radiation sensor 460 of the electronic device 140, while blocking other wavelengths from reaching the radiation sensor 460.

The detectable signals 555 and 565 overlap, as do the camera channels 565 and 570, such that in the area between approximately 550 nm and 650 nm the signal detected in either channel may include information descriptive of the other detectable signal. As such, the emitted fluorescence electromagnetic radiation 445 is characterized by a biplexed intensity distribution comprising the first detectable signal and the second detectable signal. Advantageously, the energy ranges of the passbands 575 and 580 reduce the influence of crosstalk by blocking the wavelength range between approximately 550 nm and 610 nm where the detectable signals 555 and 560 overlap. As shown, the position of the passbands 575 and 580 is a balance of transmitting the peak wavelengths of the detectable signals 555 and 560, while also avoiding crosstalk. To that end, both the extents and width of each passband is selected to provide sufficient sum intensity, integrated over the passband, while also reducing or eliminating the influence of crosstalk. The passbands 575 and 580 are included as nonlimiting examples, and it is intended that the specific energy ranges of each passband be configured based on the combination of detectable agents being employed, to selectively transmit the detectable signals being generated while also reducing crosstalk.

The example graph 550 illustrates that a portion of the emission attributed to the first detectable signal 555 does overlap the wavelength range of the second passband 580. This illustrates that the signal measured by the camera red channel 570 will include some information from the first detectable signal 555 (e.g., crosstalk). Furthermore, the second passband 580 does not include approximately half of the full width at half max of the second detectable signal 560, within the range of energies where the first detectable signal 555 overlaps the second detectable signal 560 to a larger extent. In this way, the example graph 550 illustrates the determination made to configure the passbands 575 and 580 such that signal detection is permitted while also reducing crosstalk that reduces accuracy of detection and may result in false positives. A positive result, in this context, refer to detection of emission that is attributed to the second detectable signal 560 and a generation of a differential emission signal that is positive, indicating presence of the target moiety. A false positive, therefore, refers to an assay that inaccurately determines a positive differential emission signal, for example, where the first detectable signal 555, corresponding to a positive control, is detected in the camera red channel 570 as the second detectable signal 560.

FIGS. 6A, 6B, and 6C describe the determination of normalized differential emission values as part of detecting a target moiety, such as a target nucleic acid molecule, relative to a positive control nucleic acid molecule. As described in more detail in reference to FIGS. 3-5B, detection may include optical detection of probes of amplicons of the respective nucleic acid molecules, detected through the operation of the example system 100 of FIG. 1.

FIG. 6A is an example graph 600 illustrating exemplary normalized differential fluorescence spectra of the example system 100 of FIG. 1, in accordance with one embodiment of the present disclosure. The example graph 600 includes exemplary spectra, plotted on axes of time in minutes on the abscissa and normalized differential fluorescence on the ordinate. Fluorescence is used as an illustrative example in the context of example system 100 that employs fluorophores to generate detectable signals for the control molecule and the target molecule. It is understood that stimulated emission from other detectable agents is contemplated, in other energy ranges and modalities.

The example graph 600 includes spectra for a true negative control 605, a negative sample 610, a sub-limit sample 615, a limit sample 620, a positive sample 625, and a high sample 630. The temporal dynamics illustrated in the example graph 600, principally characterized by the slope of each spectrum after a takeoff point 635 at approximately 22 min, are dependent on the specific parameters of the test protocol being employed. It is understood that the time to detection may depend on the amplification temperature, the composition of the amplification reagents, as well as other factors known in the art to affect the rate of a nucleic acid amplification process. In this way, the numerical values included in the example graph 600 are intended to illustrate the facility of the differential emission measurement approach for detecting a target moiety.

The differential emission value may be determined by the difference of the second detectable signal (e.g., second detectable signal 455 of FIG. 4 and second detectable signal 560 of FIG. 5B) and the first detectable signal (e.g., second detectable signal 450 of FIG. 4 and second detectable signal 555 of FIG. 5B). In this way, where the intensity of the first detectable signal exceeds that of the second detectable signal, the differential emission value and the test result will be negative. Conversely, where the intensity of the second detectable signal exceeds that of the first detectable signal, the differential emission value and the test result will be positive.

As illustrated in FIG. 5B, the measured intensity of each respective signal will depend on both the optical density of the emission filter (e.g., emission filter 320 of FIG. 3) and the response function of the radiation sensor (e.g., radiation sensor 460 of FIG. 4) across the relevant passband of the emission filter. For example, the camera red channel 570 illustrated in FIG. 5B demonstrates the nonuniform response of a phone camera to wavelengths in the reddish range. Furthermore, the response across relevant energy ranges may differ for the radiation sensor, such that one or more correction approaches may be applied to reduce inaccuracies introduced by the filter and/or the sensor.

For example, a correction factor may be defined whereby the specific wavelength dependence of optical density and the relative difference in sensor response are corrected, thereby permitting the detectable signals to be directly compared. In an illustrative example, a gamma correction is applied to each detectable signal, after which the first detectable signal is subtracted from the second detectable signal. Using this approach, the signal intensities attributed to each respective detectable signal may be determined over a surface of the porous matrix 115 for multiple time points to generate the spectra illustrated in the example graph 600. For example, the process of excitation and detection, as described in more detail in reference to FIG. 4, may be repeated multiple times until a statistically significant result is reached.

In the example graph 600, detectable signals are generated every 30 seconds for a total of 45 minutes, but this is not intended to be limiting. In some embodiments, the sampling rate and test duration may be selected to balance system resources with system dynamics, such that a statistically significant result is obtained while preserving system resources including, but not limited to, system power, radiation source lifetime, porous matrix condition, or throughput. With regard to statistical significance, the high sample 630 spectrum demonstrates a reduced variance relative to the positive sample 625 and the limit sample 620, as illustrated by vertical bars crossing each spectrum (e.g., a confidence interval for a given statistical technique). As shown, the time to a significant result may also be dependent on the concentration of the target moiety in a sample. The example graph 600 demonstrates, therefore, that in some embodiments, the time to a result may be less than 45 minutes. For example, the time to a significant result may be or include about 40 minutes or less, about 35 minutes or less, about 30 minutes or less, about 25 minutes or less, about 20 minutes or less, about 15 minutes or less, about 10 minutes or less, about 5 minutes or less, or less, depending on the dynamics of the system (e.g., the takeoff point 635) and/or the concentration of the target moiety in the sample.

FIG. 6B is an example graph 640 illustrating exemplary normalized differential fluorescence values of the example system 100 of FIG. 1, in accordance with one embodiment of the present disclosure. The example graph 640 includes values for: a negative control 645, a negative sample 650, a sub-limit sample 655, a limit sample 660, a positive sample 665, and a high sample 670. The statistical significance of each value is demonstrated in relation to the negative control 645 and to the negative sample 650 by either “*” or “**” representing a one-way ANOVA p-value less than two different values. For example, “*” may represent a p-value less than 0.01, while “**” may represent a p-value less than 0.003. In this way, the positive control 650, the positive sample 665, and the high sample 670 are illustrated as statistically different relative to the negative control 645 at the first “*” significance level. Similarly, the limit sample 660, the positive sample 665, and the high sample 670 are statistically different relative to positive control 645 at the “**” significance level.

FIG. 6C is a schematic diagram illustrating example porous matrices corresponding to a subset of the exemplary values of FIGS. 6A-6B, in accordance with one embodiment of the present disclosure. It is understood from the definition of the differential emission value, as described in more detail in reference to FIG. 6A, that a negative value indicates a negative result and a positive value indicates a positive result. In the context of the example system 100 of FIG. 1, the sign of the differential emission value arises from the relative intensities of the detectable signals generated by detectable agents in the porous matrix 115. The example porous matrices, as illustrated, represent a plano-rectified image of different samples as generated by a camera, through the emission filter 320 of FIG. 3. As such, the example porous matrices include a positive sample 675, a negative control 680, and a negative sample 690. In some embodiments, a substrate holder 110 may include porous matrices 115 for each sample illustrated as part of FIG. 6C, as an approach to improving detection precision and/or accuracy.

While the detectable agents (e.g., detectable agents 415 and 420 of FIG. 4) may be colocalized in the porous matrix, fluid transport and thermal dynamics may influence the uniformity of emission from the detectable agents. To that end, the positive sample 675 includes a dark region 677 and a bright region 679, where the bright region 679 is shaded to represent emission of the second detectable signal and the first detectable signal together. In contrast, the negative control 680 does not exhibit emission beyond that of the dark region 677. The negative sample 690, however, exhibits emission from the first detectable signal, but not the second detectable signal in a second bright region 691. The emission from the respective samples will result in the differential emission value being positive for the positive sample 679, being near zero for the negative control 680, and being negative for the negative sample 890.

FIG. 7 is a block flow diagram illustrating an example process 700 for detecting a target moiety in a sample, in accordance with one embodiment of the present disclosure. The process blocks of the example process 700 are described in an example order but are not intended to be in a particular order. In some embodiments, the example process 700 may omit one or more operations, may repeat one or more operations for an unspecified number of iterations, or may execute one or more operations in parallel. In some embodiments, the example process 700 is a computer-implemented series of operations executed in accordance with computer readable instructions stored on a non-transitory computer readable memory. The example process 700 is an example of the operations undertaken by components of the example system 100 of FIG. 1.

In some embodiments, the operations of the example process 700 may be preceded by one or more optional operations including, but not limited to, preparation, sampling, and/or loading of the porous matrix 115. Furthermore, the operations of the example process 700 may be preceded by initialization of a diagnostic procedure using the components of the example system 100. For example, a sample may be collected from a subject to check for a target moiety (e.g., MRSA), which may be prepared for loading into the porous substrate 115 (e.g., by suspension in a solvent and loading into a pipette), after which the prepared sample may be injected into the sample holder 110. The sample holder 110 may activate internal systems, as described in more detail in reference to FIG. 2, to convey the sample to the porous matrix 115 and to heat the porous matrix 115. Heating the porous matrix may include heating to a temperature and for a period of time sufficient to amplify the target moiety (e.g., a target nucleic acid) above a limit of detection, wherein the limit of detection corresponds to a differential emission value greater than zero.

At process block 705, the example process 700 includes generating excitation electromagnetic radiation. In some embodiments, generating excitation electromagnetic radiation includes activating the radiation source 405. As described in more detail in reference to FIG. 4, the radiation source may be or include an LED source, a halogen source (e.g., a Xenon lamp), or another source of broad-spectrum radiation. In some embodiments, the radiation source generates visible spectrum photons, as illustrated in example spectrum 500 of FIG. 5A. To improve the precision and quality of the diagnostic being performed, the excitation electromagnetic radiation may be filtered using the excitation filter 310, thereby preventing photons in the same or similar energy range as the detectable signals 450 and 455 from leaving the housing 130.

Subsequent generating the excitation electromagnetic radiation, the example process 700 includes detecting the emission 445 from detectable agents 415 and 420 in the porous matrix 115 at process block 710. Detecting emission may include activating the radiation sensor 460 and capturing sensor signals including the detectable signals 450 and 455. Advantageously, filtering the emission through the emission filter 320 blocks photons from other sources from reaching the radiation sensor 460, thereby improving the accuracy of the example process 700. The radiation sensor 460 may be or include a camera and/or a photodiode configured to receive incident radiation in an energy range corresponding to the detectable signals 450 and 455 and to convert the radiation into an electrical signal.

Subsequent detecting the emission 445, the example process 700 includes determining a differential emission value at process block 715. Determining the differential emission value may include one or more corrections to the signal generated at process block 710. For example, process block 715 may include applying a gamma correction to each energy channel of the radiation sensor 460 to control for non-uniform sensitivity across energy ranges corresponding to the detectable signals 450 and 455. In this way, determining the differential emission value includes subtracting the normalized intensity of the first detectable signal 450 from the normalized intensity of the second detectable signal 455. The intensity of each respective signal may be determined in one or more approaches. For example, in the context of a camera, the intensity in each respective camera channel may be measured on a pixel-wise basis for the region of the image including the porous matrix 115. The intensity values may be processed statistically to determine a mean value and a variance, from which a confidence interval may be defined. In some embodiments, each respective detectable signals is integrated across the porous matrix 115 by optics 325 interposed between the porous matrix 115 and the radiation sensor 460. For example, where the radiation sensor includes photo-diodes calibrated to detect the detectable signals 450 and 455, lenses (e.g., a condenser lens and an objective lens) may be used to gather the emission from the porous matrix 115 onto the radiation sensor 460.

Subsequent determining the differential emission value, the example process 700 includes comparing the value against a detection limit at decision block 720. The comparison may include statistical analysis (e.g., one-way ANOVA) to determine whether the measured value is statistically distinguishable from a negative control. Where the differential emission value cannot be distinguished from the negative control, the example process may iterate the preceding operations of process blocks 705, 710, and 715, to generate a new differential emission value. In some embodiments, the iteration is delayed, for example, by about thirty seconds. In some embodiments, the example process 700 is time-limited, such that new differential emission values are generated iteratively until the procedure time has elapsed.

At decision block 725, the sign of the differential emission value is determined. Where the differential emission value is positive, the example system 100 outputs a positive result at process block 730. Where the differential emission value is negative, the example system 100 outputs a negative result at process block 735. Outputting, as described in more detail in reference to FIG. 1, may include one or more visualizations generated via a display (e.g., display 145 of FIG. 1), notifications, and/or communicating data to one or more computing systems (e.g., a data store, a remote server, or a user device). The data may be or include the result, derived data, and/or raw data.

### Example 1—Two-Fluorophore Imaging of Biplexed Real-Time Nucleic Acid Amplification Tests

The following paragraphs describe an example embodiment of the system described in reference to FIGS. 1-4. It is understood that the detailed implementation, components, operation, and features of the example below are non-limiting and that embodiments of the present disclosure may include alternative components, operations, and/or features than those described.

Imaging point-of-care assays with mobile phones presents several advantages due to phones' ubiquity, low cost, ease of use, and breadth of integrated hardware. These advantages are predicated on either leveraging end users' mobile phones (e.g., for home use) or manufacturing low-cost, near-disposable assay readers that integrate the low-cost camera and communications hardware used in mobile phones. However, most of the mobile phone-imaged assay literature demonstrates compatibility with just one camera sensor module or product line, even for assays imaged by multiple mobile phone models. Indeed, many assays take advantage of a quirk of a particular phone model, such as a xenon lamp camera flash or atypically long exposure time, to improve assay performance. In contrast, ratio-metric imaging enables highly sensitive fluorescence measurements without optical filters on multiple mobile phone models, including phones with differing camera spectral sensitivities, by exciting large Stokes shift fluorophores with ultraviolet light-emitting diodes (LEDs). However, there remains a need in the literature to image standard fluorophores with small Stokes shifts by using multiple mobile phone models with appropriate integrated controls.

Integrated positive controls enhance assay sensitivity by discriminating between putative negative results and assay failure (thereby reducing false negatives). For example, real-time NAATs may include additional fluorescent probes that are detected in an orthogonal detection channel to monitor non target background amplification products. In contrast, many point-of-care assays spatially separate such positive controls from the signal of interest and use the same optical detection strategy (e.g., visual readout with chromophore-labeled lateral flow strips) to detect both the target and positive control. However, real-time NAATs cannot always rely on spatially separated positive controls: false negatives are possible when amplification fails in the region corresponding to the signal of interest even if amplification occurs in the positive control region (e.g., due to heater or fluidic failure).

In one embodiment, a hardware and signal processing system images two colocalized fluorophores with mobile phones, which enables real-time, point-of-care NAATs. Multi-pass excitation and emission filters are attached to the flash and camera of multiple mobile phone models, permitting the mobile phones to image fluorophores at NAAT-relevant concentrations in optically scattering media such as paper. It was observed that evaporation during amplification introduces an optical artifact that was overcome with a differential optical signal processing scheme. Finally, these insights were applied to a multiplexed, disposable NAAT (“MD NAAT”) platform powered by a single printed circuit board (PCB), wherein biplexed isothermal strand displacement amplification reactions in real time were imaged. These results suggest a path toward real-time fluorescence NAATs in the home.

As an overview of the proposed nucleic acid amplification test (NAAT) fluorescence reader, typical NAATs with endpoint detection (e.g., with lateral flow strips) include discrete sample preparation, amplification, and detection steps. A strategy to reduce time to result includes detecting amplifying nucleic acids in real time with a two-fluorophore mobile phone reader, which were validated by imaging the “MD NAAT” platform.

In the example embodiment, a two-fluorophore mobile phone reader images biplexed NAATs amplifying in real time. The reader detects green and red fluorophores such as fluorescein and Texas Red, which can both be excited with a broadband light-emitting diode (e.g., phone flash coupled to a multipass excitation filter) and simultaneously, but separately, detected with a standard smartphone camera (with a multipass emission filter). The LED of the phone's flash emits white light, which is filtered by the excitation filter and excites fluorescein and Texas Red. The fluorescence emission then passes though the emission filter and is detected by the phone's camera, further illustrated in FIG. 4. Fluorescence emission from fluorescein is detected in the green color channel of the phone's image sensor, while emission from Texas Red is detected in the red color channel of phone's image sensor due to the Bayer mosaic filter on the camera sensor. FIGS. 5A-B show the spectra of the phone LED, filters, and camera photo color channels. In some embodiments, the differential fluorescence signal between the red and green color channels is defined by the expression:

Differential fluorescence signal=Red intensity_{γ-corrected}−Green intensity_{γ-corrected} (1)

Design of the Mobile Phone Fluorescence Reader

Housings for two common mobile phones were designed by layering 1/16″ (1.59 mm) and ⅛″ (3.18 mm)-thick PMMA sheets that were laser cut, assembled, and then chemically welded with dichloromethane. The filter holder design was engineered to minimize light leakage (including autofluorescence) with contours corresponding to the phones' profiles. Cutouts over the camera aperture and camera flash LED hold dual-bandpass 479/585 nm excitation and 524/628 nm emission filters for fluorescein and Texas Red, respectively. The excitation light is partially collimated with a 12 mm-wide, 12 mm focal length plano-convex lens placed 6 mm from the light-emitting diode.

Estimating Limits of Detection

Limits of detection of the mobile phone fluorescence reader were estimated at conditions mimicking that of a NAAT run. Dilution series of fluorescein or Texas Red were created in a mock isothermal strand displacement amplification (iSDA) solution at concentrations of 0, 100, 150, 200, 250, 300, or 400 nM. The diluent contained magnesium sulfate (3.75 mM), trehalose (270 mM), dextran (500 kDa, 4.8% w/v), and anhydrous glycerol (0.41% (v/v)) in phosphate-buffered saline (10 mM, pH=7.4). Glass fiber pads were laser cut, placed into laser ablated PMMA cartridges, and rehydrated with 20 μL of fluorophore solution. The cartridges were mounted to the printed circuit board of an MD NAAT platform with thermally-conductive tape. Pads were heated for about three minutes and imaged with the mobile phone reader Images were acquired at ISO 1600, 0.25 sec. exposure, incandescent white balance, gamma-corrected, quantified in the red and green color channels, and statistically analyzed.

Characterizing Evaporation Artifact

The mobile phone reader observed the characterization of how evaporation in heated pads (e.g., during amplification) affects the fluorescence intensity. Since the glass fiber pads are an optically scattering substrate, the refractive indices of the fibers of the glass fiber membranes were measured to characterize the impact of light scattering on observed fluorescence intensities. Samples of glass fiber were punched to 15.9 mm diameter circles and mounted in a refractometer to measure the refractive index at 589.3 nm and 20.0° C. Several liquid controls were measured, including 100 μL of immersion oil of known refractive index (1.460, 1.480, and 1.500 at 589.3 nm) or 500 μL of: nuclease-free water, p-xylene, and simulated NAAT master mix (water mixed with trehalose (270 mM) and 500 kDa dextran (4.8% w/v)). Since the glass fiber pads were heterogeneous (especially as used in an assay when blocked with bovine serum albumin), it was verified whether the measured refractive indices were correct by imaging pads that were soaked with an index-matching liquid. Brightfield images were captured showing glass fiber pads blocked with bovine serum albumin (procedure below) that were wetted with 20 μL of water (as a control) or p-xylene (which index-matches).

A measurement was taken of how observed fluorescence intensities changed during a simulated NAAT run. Glass fiber pads were blocked with bovine serum albumin (1% w/v) and Tween-20 (0.1% v/v), mounted in PMMA cartridges as before, and heated for 60 minutes in the MD NAAT device. A Nexus 5×-based mobile phone fluorescence reader was mounted about 15 cm above the MD NAAT and imaged the pads about every 30 seconds (ISO 3200, 0.25 sec. expo-sure, incandescent white balance). Images were gamma-corrected and quantified in the red and green color channels.

Applying Fluorescence Reader to Image a NAAT in Real Time

A validation of the ability of the mobile phone reader to image real-time nucleic acid amplification in the MD NAAT device was undertaken. Isothermal strand displacement amplification (iSDA) targeting the ldh1 or mecA genes of Staphylococcus aureus and an internal amplification control (IAC) was performed. Glass fiber pads were laser-cut, blocked, filled with all iSDA reagents including enzymes, primers, and probes, and subsequently flash-frozen and lyophilized. The lyophilized pads were placed in PMMA cartridges and rehydrated with samples containing genomic DNA from methicillin-resistant S. aureus (MRSA) strain FPR3757 and/or 100 k copies of IAC single-stranded DNA in nuclease-free water. Filled cartridges were sealed with PCR tape, adhered to the MD NAAT PCB with thermally conductive tape, and heated over USB power by the MD NAAT device for 45 minutes (set point 55.3° C., Kₚ=65535, Kᵢ=10).

Amplification reactions were imaged by a mobile phone equipped with multipass excitation and emission filters about every 30 seconds. The images were analyzed by cropping them to the glass fiber pads, normalizing the individual color channel intensities, and gamma correcting. The differential fluorescence intensity was calculated for each pixel based on expression (1), above and averaged across each glass fiber pad.

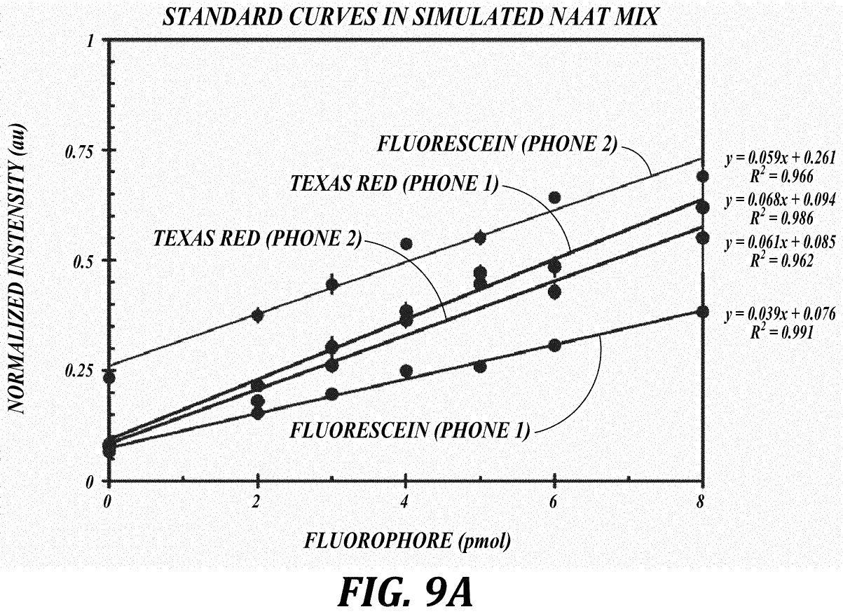

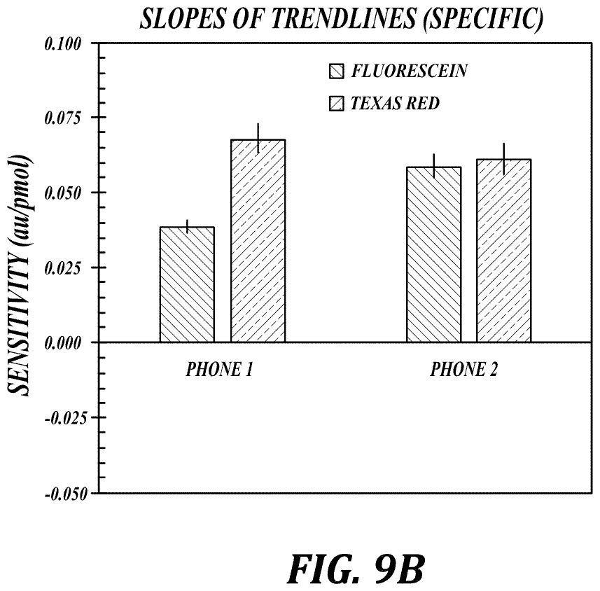

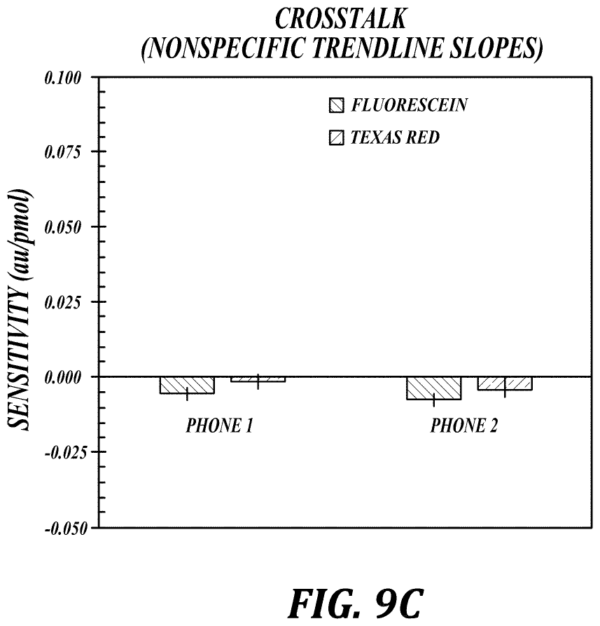

FIG. 9A and FIG. 9B illustrates a design of a mobile phone fluorescence reader that images two colocalized fluorophores, such as in point-of-care NAATs, in real time. FIG. 4 depicts a cartoon of the mobile phone fluorescence reader shows how dual-bandpass excitation (exc.) and emission (em.) filters image colocalized fluorophores simultaneously. FIG. 3 depicts a rendering and FIG. 8A depicts a photo of the fluorescence reader. FIG. 8B depicts a photo of 49 spots of fluorophore mixtures of Texas Red and fluorescein on glass fiber to show a phone's ability to image fluorophores at NAAT-relevant concentrations (200 nM) over a large 5×5 cm field of view (2nd row and 3rd column from left and top). Noncircular spot patterns are due to wicking in the heterogeneous glass fiber membrane.

Hardware Validation and Limits of Detection

The mobile phone reader was validated by imaging dilution series of two fluorophores with two mobile phones. First, the ability of the mobile phone reader to image a large (5×5 cm) field of view was confirmed by imaging a five-fold dilution series of Texas Red and fluorescein in phosphate-buffered saline (pH 7.4) from 20 μM to 6.4 nM (and negative controls) in all combinations of Texas Red and fluorescein that had been pipetted onto a porous membrane (10 μL per spot). Fluorophores were visible at levels relevant to the NAAT fluorescent probe levels (200 nM) as suggested by visible spots in the 2^{nd} and 3^{rd} columns and rows of FIG. 8B.

Next, the fluorophore limits of detection of the phones were determined at NAAT-relevant conditions (FIG. 9A). Glass fiber pads filled with fluorophore and mock iSDA master mix were heated in the MD NAAT and imaged with two phones. Both phones detected fluorescein in the green color channel and Texas Red in the red color channel (Pearson product-moment correlation coefficient: r>0.98, p<0.001, t-test). Texas Red detected in the red color channel showed limits of detection of 1.2 pmol and 1.7 pmol for two different phones, while fluorescein detected in the green color channel had LODs of 0.64 pmol and 3.7 pmol, respectively. The observed limits of detection were lower than the nominal fluorescent probe amounts during a NAAT run (4.0 pmol). There was no significant difference in the sensitivity to Texas Red or fluorescein for both phones (p>0.7).

Crosstalk was measured by assessing to what extent fluorescein was detected in the red color channel and Texas Red in the green color channel There was no correlation between Texas Red concentration and fluorescence in the green color channel on both phones as indicated by the near-zero slopes and correlation coefficients in FIG. 9B (r=0.4, p>0.2). However, there was a slight negative correlation between fluorescein concentration and fluorescence in the red color channel as indicated in FIG. 9C (r=−0.8, p=0.01), albeit with a small effect size (the slope in the red color channel was −⅛ the slope in the green channel).

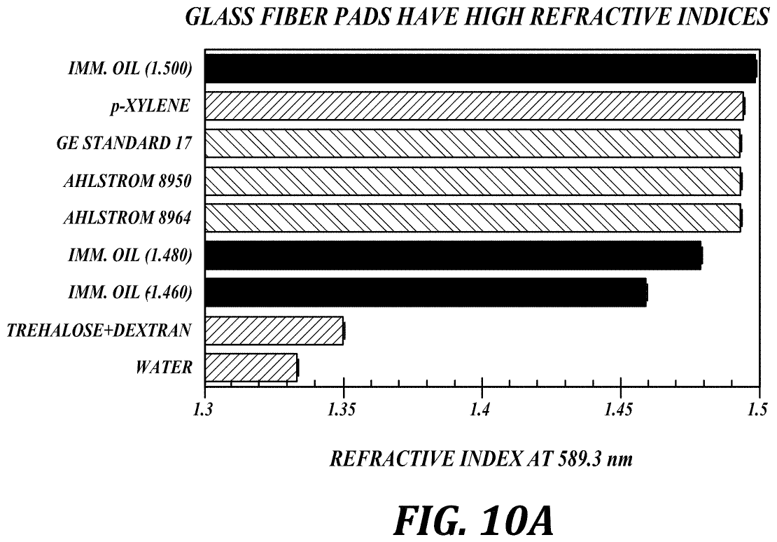

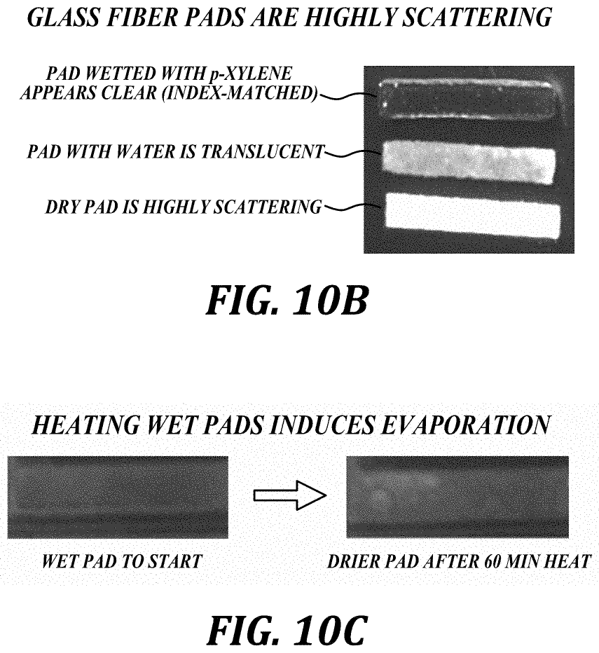

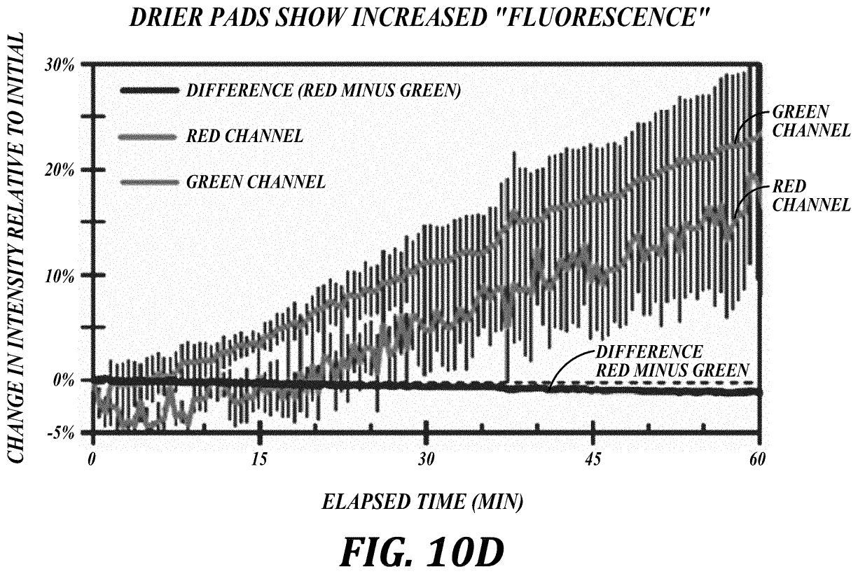

FIGS. 10A-D illustrate imaging fluorophores in high surface area, optically scattering substrates such as paper requires overcoming a light scattering artifact driven by evaporation. FIG. 10A illustrates the measured refractive indices of the fibers of several glass fiber materials (orange bars) at about 1.4931, which was greater than that of water (1.33285) or simulated NAAT master mix (trehalose+dextran, 1.35019) at 589.3 nm and 20° C. The gray bars indicate reference immersion (imm) oil controls. Bars indicate mean and error bars show 95% confidence intervals (too small to see in some cases). FIG. 10B illustrates a glass fiber pad blocked with bovine serum albumin that was wetted with index-matching p-xylene appeared transparent, while a pad wetted with water was translucent, and a dry pad scattered light and appeared white. FIG. 10C illustrates glass fiber pads blocked with bovine serum albumin that were wetted with water showing evaporation and condensation after 60 minutes of heating (to 50° C.) when imaged with the two-fluorophore mobile phone reader. FIG. 10D illustrates quantifying the heated pads' fluorescence intensity showing that the red and green color channel intensities increased irreproducibly as the pads were heated, but the difference of the two color channels consistently decreased by only 1%. Lines show mean and standard error of the mean (n=4 pads).

The effect of evaporation on imaging was then characterized. First, the refractive index of the glass fiber substrate which supports nucleic acid amplification was measured. FIG. 10A shows that the refractive index of the glass fiber substrate was 1.49308±0.00003 (mean and 95% confidence interval) at 589.3 nm and 20° C., which is similar to that of other glass fiber materials and p-xylene (1.49444±0.00004), but substantially higher than the refractive index of water (1.33285±0.00000). FIG. 10B shows that a BSA-blocked glass fiber pad wetted with an index-matching liquid such as p-xylene is nearly transparent, whereas a pad wetted with water is translucent and a dry pad appears uniformly white.

The fluorescence intensities of wetted glass fiber pads was measured during a simulated NAAT. Glass fiber pads filled with mock NAAT master mixes without any fluorophores were heated for one hour in the proposed device and imaged with the mobile phone reader in real time (FIGS. 10A-D). The observed intensities at 45 minutes increased by 11±6% (mean and standard error of the mean) in the red channel and 17±5% in the green channel relative to the initial level. In contrast, the difference of the red and green color channels (based on expression (1)) decreased by 0.92±0.18% relative to the initial level, which was significantly below the threshold for positive (0.02 units, p<0.001 at all times, left-tailed one-sample t-test) and above the threshold for negative (−0.02 units, p<0.01 at times at or below 45 minutes, right-tailed one-sample t-test). In other words, the differential fluorescence signal was within the range corresponding to no amplification for amplification times up to 45 minutes.

FIGS. 6A-C illustrate exemplary data characteristic of biplexed isothermal strand displacement amplification reactions performed in the MD NAAT platform with a user device demonstrates the utility of the differential fluorescence imaging strategy. In this exemplary embodiment, glass fiber pads contained lyophilized reagents for isothermal strand displacement amplification, including the template-specific fluorescent probes labeled with fluorescein (which detected internal amplification control (IAC) DNA) and a Texas Red analog (which was specific to MRSA DNA). FIG. 6A illustrates exemplary data characteristic of pads rehydrated with mixtures of MRSA genomic DNA and internal amplification control (IAC) DNA showing rapid amplification with liftoff near 30 minutes. Biplexed samples containing methicillin-resistant S. aureus (MRSA) genomic DNA and internal amplification control (IAC) DNA show positive signals (i.e. more amplification in the MRSA-specific red channel than IAC-specific green channel), while samples containing only IAC show negative signals (i.e. more signal in the green channel than red). Truly negative samples containing neither MRSA nor IAC DNA show near-zero signals (i.e. nearly equal signal in both the red and green channels). Each curve includes mean and error bars showing standard error of the mean (n=3). FIG. 6B shows exemplary data characteristic of the differential fluorescence at 45 minutes. In this example, significant differences were detected between the true negative control and the IAC only case (*p<0.01, one-way ANOVA), the true negative and samples containing at least 1 k copies of MRSA (*p<0.01, one-way ANOVA), and the IAC only case and samples containing at least 316 copies of MRSA (**p<0.003, one-way ANOVA). Bars show mean and error bars show 95% confidence intervals. FIG. 6C illustrates exemplary diagrams characteristic pads at 45 minutes that, in this example embodiment, showed substantial red fluorescence in pads containing 1 k copies of MRSA gDNA and 100 k copies of IAC DNA, while pads containing only IAC showed substantial green fluorescence and pads containing neither did not fluoresce appreciably.

Application to Real-Time NAATs

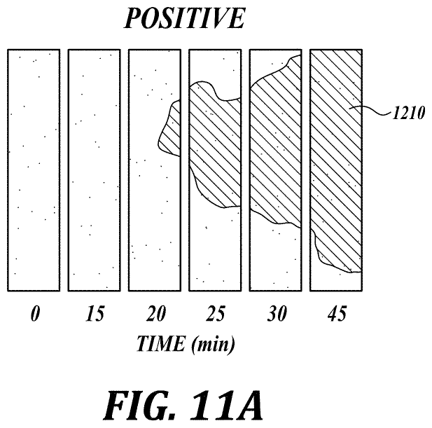

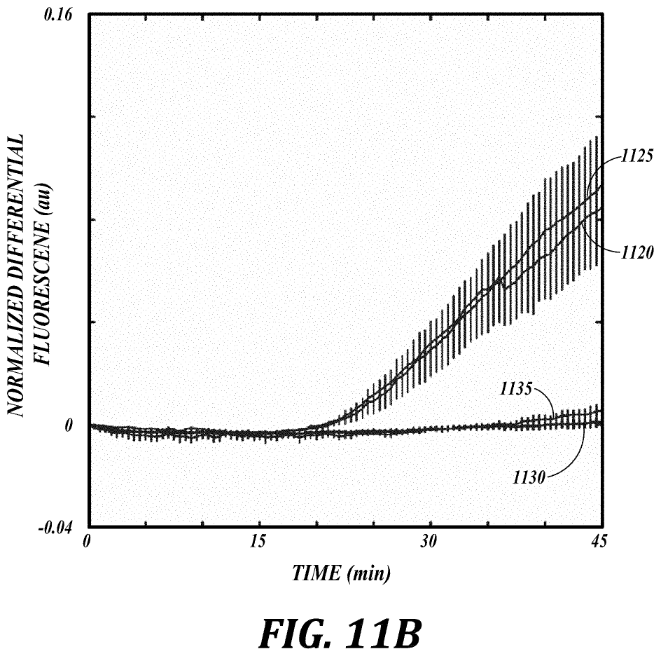

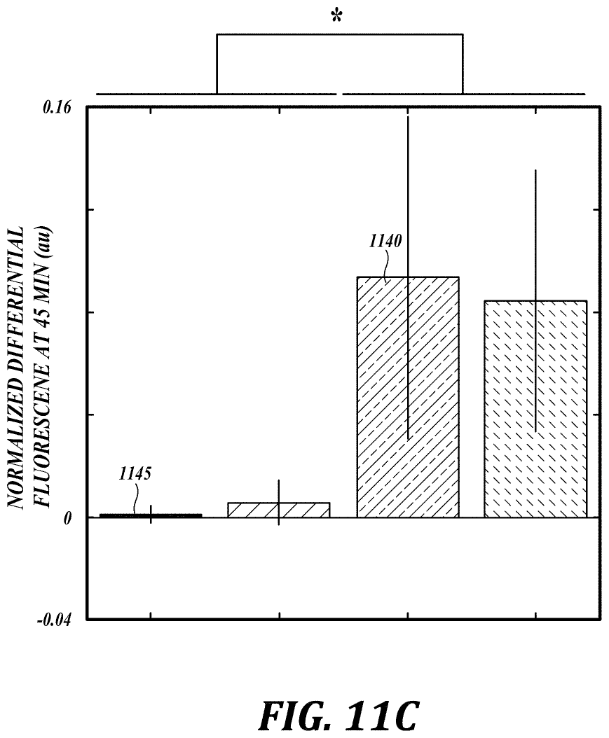

In this example embodiment, isothermal strand displacement amplification (iSDA) reactions were imaged in the MD NAAT platform in real time with two phones. Both singleplexed and biplexed reactions were run, targeting different genes in methicillin-resistant S. aureus (FIGS. 11A-C and FIGS. 6A-C). Mixtures of MRSA genomic DNA (gDNA) and/or internal amplification control (IAC) DNA templates in were prepared buffer, rehydrated glass fiber pads containing lyophilized iSDA reagents, heated the pads in the MD NAAT platform, and imaged with a phone equipped with multipass excitation and emission filters every 30 seconds. Both experiments were quantitatively analyzed by considering the normalized differential fluorescence as defined in expression (1); FIG. 6B indicates how to interpret these data.

FIG. 6A depicts standard curves of glass fiber pads containing fluorescein or Texas Red imaged by the mobile phone fluorescence reader. Pads were saturated with mock nucleic acid amplification reagent mixtures that mimic the pH, salt, and viscosity of iSDA and imaged at iSDA temperatures. Limits of detection for both fluorophores are lower than the nominal fluorescent probe amounts (4.0 pmol) in each iSDA reaction (Nexus 5×: 1.2 pmol (Texas Red) and 0.64 pmol (fluorescein) and Pixel 2: 1.7 pmol (Texas Red) and 3.7 pmol (fluorescein)). Points indicate mean and standard error of the mean (n=4). FIG. 6B depicts both user devices being similarly sensitive to fluorescein and Texas Red (as indicated by the slope of the linear regression of fluorescein in the green channel and Texas Red in the red channel, p>0.7). FIG. 6C depicts minimal crosstalk as suggested by the near-zero slope in the nonspecific color channels (fluorescein in the red color channel and Texas Red in the green color channel).

FIGS. 11A-C depict imaging singleplexed amplification reactions in the MD NAAT in real time with the user device, such as a Nexus 5× phone. Glass fiber pads containing lyophilized isothermal strand displacement amplification reagents (with a red-emitting fluorescent probe for mecA) were rehydrated with samples containing MRSA genomic DNA or water. Pads were heated in the MD NAAT for 45 minutes and imaged about every 30 seconds with a Nexus 5× equipped with multipass excitation and emission filters for fluorescein and Texas Red. FIG. 11A depicts schematic illustrations of an amplifying pad 1200 showing substantial fluorescence in a MRSA-containing pad in bright region 1210 that grows over time. FIG. 11B depicts real-time curves show the average differential fluorescence intensity across each pad (red color channel minus green channel) over time.

The curves show rapid amplification of five hundred copies 1220 and one thousand copies 1225 of MRSA. Vertical lines show mean and standard error of the mean (n=3). In contrast, the negative control 1230 and sub-limit sample 1235 curves maintain a near-zero differential value across the period of time sampled. FIG. 11C depicts the endpoint differential fluorescence at 45 minutes is significantly higher in pads containing at least five hundred copies 1240 of MRSA than in the negative control 1245 (*p<0.001, one-way ANOVA). Bars show mean and error bars show 95% confidence intervals.

First, singleplexed iSDA reactions labeled with a red-emitting fluorescence probe specific to the mecA gene were imaged with a user device, but lacking IAC templates, probes, or fluorescent labels (e.g., FIGS. 11A-C). Images of pads (e.g., FIG. 11A) and real-time curves (of the differential fluorescence as defined in expression (1)) during amplification consistently showed negligible fluorescence, while pads containing at least 500 copies of MRSA gDNA showed substantial red fluorescence from about 20 minutes onward. The endpoint differential fluorescence for pads containing at least 500 copies of MRSA gDNA was significantly greater than the true negative control (p<0.001, one-way ANOVA, n=3 for positives and n=9 for true negatives).

Next, biplexed iSDA reactions labeled with a red-emitting probe specific to the ldh1 gene and a green-emitting probe specific to the IAC amplicons were imaged. It was observed that the real-time amplification curves showed liftoff times of about 30 minutes (e.g., FIG. 6A). In the example embodiment, pads containing high levels of MRSA gDNA showed positive differential fluorescence signals, whereas pads containing only IAC or 100 copies of MRSA gDNA showed negative differential fluorescence signals. Biplexed reactions containing at least 1000 copies of MRSA gDNA showed significantly higher endpoint differential fluorescence than the true negative control (p<0.01, one-way ANOVA), while pads containing only IAC (i.e. MRSA-negative pads) showed lower endpoint differential fluorescence than either the true negative control (p<0.01, one-way ANOVA) or pads containing at least 316 copies of MRSA gDNA (p<0.003, one-way ANOVA). In other words, pads containing only IAC had lower (more negative) levels of differential fluorescence whereas pads containing high levels of MRSA had higher (more positive) differential fluorescence levels.

Laboratory nucleic acid amplification tests are sensitive, specific, and rapid assays that combine exponential amplification, template-specific real-time fluorescence detection, and multiplexing (e.g., with onboard positive controls). For example, some techniques provide results in about 66 minutes (for S. aureus), detects up to six fluorophores, and includes onboard lysis and amplification controls. Understandably, such systems are complex and unsuitable for point-of-care deployment. This example embodiment describes a mobile phone imaging strategy that images two colocalized fluorophores simultaneously, which enables real-time fluorescence detection in biplexed NAATs. The strategy was applied to image a point-of-care, paper-based NAAT on our lab's MD NAAT platform.

Imaging fluorophore-labeled NAATs in porous media (e.g., the glass fiber pads used in this example embodiment) is challenging, at least because the substrate introduces optical artifacts due at least in part to heterogeneity, light scattering, and auto-fluorescence. Beyond these drawbacks, which are already documented in the literature, highly variable optical artifacts such as drift in the fluorescence intensities of heated glass fiber pads were also observed. It was hypothesized that this artifact was due to evaporation and condensation of liquid on the cover due to the presence of a small air gap between the pads and cover (e.g., transmission window 230 of FIG. 2). This hypothesis is substantiated by the liquid droplets in the heated pad photo in FIG. 10C. Evaporation was likely exacerbated by the pads' high surface area (45 mm²) and elevated temperature during heating (e.g., 50° C. for 45 minutes during iSDA). The artifact was not due to a temperature-dependent change in the fluorophore quantum yield during amplification because the artifact was observed even when fluorophores were not present (e.g., in FIGS. 10C-D). The rise in fluorescence intensity was attributed to increased light scattering as the glass fiber pads dried, as shown in FIG. 10B.

Differential fluorescence measurements are one strategy to overcome the artifacts imposed by the substrate's auto-fluorescence, light scattering, and saturation-dependent drift in fluorescence intensity. Differential measurements reduce drift when noise contributors appear about equally in multiple channels, such as in radar or spectroscopy. In this example embodiment, both the red and green color channels of mobile phone photos contained artifacts introduced by evaporation and paper auto-fluorescence. Analyzing the difference of the red and green color channels eliminated the impact of these factors as suggested by FIG. 3D. The usefulness of this approach was demonstrated on phones with completely different camera spectral sensitivities (e.g., spectra 565 and 570 of FIG. 5B), which suggests the generalizability of the proposed differential fluorescence measurement strategy.

One concern when imaging multiple fluorophores is crosstalk between ostensibly orthogonal detection channels. Although minimal detection of Texas Red in the green color channel was observed, it was observed that the red color channel fluorescence was slightly negatively correlated with fluorescein concentration. The practical consequence of this is potentially lower sensitivity toward the target labeled with Texas Red, or MRSA in in this example embodiment. However, sensitivity toward red fluorophore-labeled MRSA (316 copies) permitted detection in biplexed reactions in which IAC was present at 100 k copies (e.g., FIGS. 6A-C). Further improvements to sensitivity (and a practical assay's positive predictive value) may be possible with algorithmic improvements such as principal component analysis, use of additional sample volume to increase template copy numbers, and optimizing the choice of fluorophores (e.g., increased quantum yield, decreased time in the excited state, or reduced photobleaching).

The imaging scheme described in this example embodiment improves on alternatives described in the literature. Advantageously, the techniques described herein do not rely on long Stokes shift fluorophores, such as quantum dots. Furthermore, Förster resonance energy transfer (FRET) for visualizing nucleic acid hybridization in paper provides an endpoint assay that does not monitor real-time amplification. Furthermore, multiplexed mobile-phone imaging of fluorescence NAATs in nonscattering media such as tubes are unable to detect multiple fluorophores at the same time because each fluorophore required distinct emission filters, rendering the system impractical for real-time imaging in scattering media.

In this way, a mobile phone camera sensor-agnostic imaging strategy may be employed to visualize, in real time, biplexed NAATs performed in highly scattering media such as paper. Differential fluorescence imaging with phones may enable translating sensitive laboratory fluorescence assays to the point of care.

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the Figures, can be arranged, substituted, combined, separated, and designed in a wide variety of different configurations, all of which are explicitly contemplated herein.

Furthermore, the particular arrangements shown in the Figures should not be viewed as limiting. It should be understood other embodiments may include more or less of each element shown in a given Figure. Further, some of the illustrated elements may be combined or omitted. Yet further, an example embodiment may include elements not illustrated in the Figures. As used herein, with respect to measurements, “about” means +/−5%.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

1. A system, comprising:
a substrate holder comprising a porous matrix, the porous matrix comprising:
a first detectable agent configured to selectively couple to a first target moiety and to emit a first detectable signal upon fluorescence of the first detectable agent; and
a second detectable agent configured to selectively couple to a second target moiety and to emit a second detectable signal different than the first detectable signal upon fluorescence of the second detectable agent;
a housing, optically coupled with the substrate holder, and shaped to optically couple with a radiation source and a radiation sensor and to optically isolate the radiation source and the radiation sensor;
an excitation filter, disposed in or on the housing, configured to receive excitation electromagnetic radiation from the radiation source and to transmit a first portion of the excitation electromagnetic radiation to the porous matrix; and
an emission filter, disposed in or on the housing, configured to receive emitted fluorescence electromagnetic radiation from the porous matrix and to transmit a second portion of the emitted fluorescence electromagnetic radiation, the second portion being different from the first portion.

2. The system of claim 1, wherein the excitation filter is a multiple-passband filter, and wherein the first portion comprises two non-contiguous excitation energy ranges corresponding to a first excitation wavelength range of the first detectable agent and a second excitation wavelength range of the second detectable agent, respectively.

3. The system of claim 1, wherein the emission filter is a second multiple-passband filter, and wherein the second portion comprises two non-contiguous emitted fluorescence energy ranges corresponding to the first detectable signal and the second detectable signal, respectively.

4. The system of claim 3, wherein the two non-contiguous emitted fluorescence energy ranges comprise a first band in a wavelength range from about 500 nm to about 550 nm and a second band in a range from about 600 nm to about 650 nm.

5. The system of claim 1, wherein the excitation electromagnetic radiation is characterized by a continuous emission intensity distribution within a wavelength range from about 400 nm to about 700 nm.

6. The system of claim 1, wherein the emitted fluorescence electromagnetic radiation is characterized by a biplexed intensity distribution comprising the first detectable signal and the second detectable signal.

7. The system of claim 1 wherein the porous matrix further comprises reagents to amplify a target nucleic acid molecule and a positive control nucleic acid molecule.

8. The system of claim 7, wherein the first detectable agent is a probe of an amplicon of the positive control nucleic acid molecule and wherein the second detectable agent is a probe of an amplicon of the target nucleic acid molecule.

9. The system of claim 1, wherein the porous matrix comprises non-woven glass fiber.

10. The system of claim 1, wherein the first detectable agent and the second detectable agent are colocalized on the porous matrix.

11. The system of claim 1, further comprising an electronic device, wherein the electronic device comprises the radiation source and the radiation sensor, and wherein the excitation filter and the emission filter are positioned to optically couple with the radiation source and the radiation sensor, respectively.

12. The system of claim 11, wherein the electronic device is a smart phone, wherein the radiation source is an electronic flash, and wherein the radiation sensor is a camera.

13. The system of claim 11, further comprising a controller including one or more processors and a non-transitory computer readable memory storing executable instructions that, when executed by the one or more processors, cause the one or more processors to execute operations comprising:
generating the excitation electromagnetic radiation using the radiation source, the radiation source being optically coupled with the excitation filter to transmit the first portion of the excitation electromagnetic radiation to the porous matrix;
detecting the first detectable signal and the second detectable signal using the second portion of the emitted fluorescence electromagnetic radiation received by the radiation sensor via the emission filter; and
determining a differential emission value using the first detectable signal and the second detectable signal.

14. The system of claim 13, wherein determining the differential emission value comprises:
generating a first gamma-corrected signal based on the first detectable signal and a second gamma-corrected signal based on the second detectable signal; and
determining a difference between the second gamma-corrected measurement and the first gamma-corrected measurement.

15. The system of claim 13, wherein the substrate holder further comprises an electronic heating circuit thermally coupled with the porous matrix, wherein the electronic heating circuit is configured to heat the porous matrix.

16. The system of claim 15, wherein the non-transitory computer readable memory stores further instructions that, when executed by one or more processors of the system, cause the one or more processors to execute operations comprising:
heating the porous matrix to a temperature and for a period of time sufficient to amplify a target nucleic acid above a limit of detection, wherein the limit of detection corresponds to a differential emission value greater than zero.

17. A computer-implemented method for detecting a target moiety in a sample, the method comprising:
generating excitation electromagnetic radiation using a radiation source, the radiation source being optically coupled with an excitation filter to transmit a first portion of the excitation electromagnetic radiation to a porous matrix, the porous matrix comprising nucleic acid amplification reagents, a first detectable agent, and a second detectable agent;
generating an emission signal comprising a first detectable signal from the first detectable agent and a second detectable signal from the second detectable agent using a second portion of emitted fluorescence electromagnetic radiation received at an optical sensor via an emission filter, the second portion being different from the first portion; and
determining a differential emission value using the emission signal.

18. The method of claim 17, further comprising heating the porous matrix to a temperature for a period of time sufficient to amplify a target nucleic acid above a limit of detection, wherein the limit of detection corresponds to a differential emission value greater than zero.

19. The method of claim 18, wherein the period of time is in a range from about 20 minutes to about 40 minutes, and wherein the temperature is in a range from about 300 K to about 350 K.

20. The method of claim 17, further comprising:
generating a plurality of measurements of emission, including the measurement; and
generating a plurality of differential emission intensity values, including the differential emission intensity value, using the plurality of measurements of emission.
